(19) United States
(12) Patent Application Publication
(10) Pub. No.: US 2017/0202847 A1
(43) Pub. Date: Jul. 20, 2017

(54) METHODS FOR THE TREATMENT OF TUMORS

(71) Applicants: The University of North Carolina at Chapel Hill, Chapel Hill, NC (US); The Regents of the University of Colorado, Denver, CO (US)

(72) Inventors: H. Shelton EARP, III, Chapel Hill, NC (US); Xiaodong WANG, Chapel Hill, NC (US); Stephen V. FRYE, Chapel Hill, NC (US); Douglas Kim GRAHAM, Atlanta, GA (US)

(21) Appl. No.: 15/301,893

(22) PCT Filed: Apr. 3, 2015
(86) PCT No.: PCT/US2015/024258

§ 371 Date: Oct. 4, 2016

Related U.S. Application Data
(60) Provisional application No. 61/975,567, filed on Apr. 4, 2014.

(51) Int. Cl. A61K 31/5377; A61K 31/519; A61K 45/06

(52) U.S. Cl. CPC A61K 31/5377; A61K 45/06; A61K 31/519

(57) ABSTRACT

The present invention is directed to methods of treating a tumor, in particular human tumors, including administering an effective amount of a MER tyrosine kinase inhibitor (MER TKI) to inhibit TKI signaling in a tumor. The use of a MER TKI in combination with a chemotherapeutic agent, wherein the MER TKI can be administered to a host with a cancer prior to, during, or after administration with a chemotherapeutic agent, provides for increased anti-tumor effects without an increase in the standard of care dosage of the chemotherapeutic agent.

### CROSS-REFERENCE TO RELATED APPLICATION

This application claims the benefit of U.S. Provisional Application No. 61/975,567 filed Apr. 4, 2014. The entirety of this application is hereby incorporated by reference for all purposes.

### STATEMENT OF FEDERAL SUPPORT

This invention was made with government support under Grant No HH5N26120800001E awarded by the National Institutes of Health. The Government has certain rights in the invention.

### FIELD OF THE INVENTION

This invention is in the area of improved methods for treating tumors, in particular human tumors.

### BACKGROUND

MerTK is a member of a receptor tyrosine kinase (RTK) family that also includes AXL and TYRO3. Family members undergo ligand-induced homodimerization, followed by catalytic tyrosine kinase activation and intracellular signaling. Cross-phosphorylation has also been demonstrated within this RTK family, suggesting heterodimerization. These RTKs are widely expressed in many epithelial tissues and in cells of the immune, nervous, and reproductive systems. The MerTK ligands include growth arrest-specific 6 (GAS6), protein-S, tubby and tubbylike protein-1 (TULP1), and galectin-3. Several of these ligands are present in serum, and all are expressed locally in some tissues. These ligands bind to the extracellular domain of MerTK, resulting in tyrosine kinase activation. MerTK is expressed in non-neoplastic cells found in the tumor microenvironment. MerTK is also ectopically expressed or overexpressed in many hematologic and epithelial malignant cells. Moreover, expression of MerTK and GAS6 correlates with poor prognosis or chemoresistance in some human tumor types. However, the mechanisms by which increased MerTK signaling contributes to tumor malignancy remain unknown.

It is an object of the present invention to provide new methods for the treatment of tumors.

### SUMMARY

The present invention provides new methods for the treatment of tumors using small molecule MER tyrosine kinase inhibitors (MER TKIs). In addition to the specific MER TKIs described herein, the methods of the current invention can be performed with MER TKIs described in WO2011146313, WO02013052417, WO2013177168, PCT/US2013/065192, and PCT/US2013/71409, incorporated herein in their entirety.

MER TKIs have dual anti-cancer effects. MER TKIs are capable of direct anti-cancer effects by inhibiting MER tyrosine kinase within tumor cells, which acts as a survival signal for tumors, and the inhibition thereof can result in the reversal of survival and chemoresistance in tumor cells.

It is known that tumor associated macrophages in the tumor microenvironment can aid the survival of a tumor (“tumor immunity”) by expressing cytokines that inhibit the natural immune response. MER TKIs can also act by inhibiting MER TK in the host macrophage which results in the suppression of tumor immunity and increased immune responses against tumor cells.

It has been surprisingly discovered that these two activities of MER TKIs can be separated, and that at a low dose (for example, approximately 1-100 mg/dose), MER TKIs can exhibit an immunotherapeutic effect only. By taking advantage of the differences in MER TKI activities based on the dosage required to induce the two effects, one can optimize tumor therapy. In one embodiment, a method for the treatment of a tumor is provided that includes administering an effective amount of a MER TKI to inhibit TKI signaling in a tumor associated macrophage, without inhibiting the survival signal in the tumor itself. In this way, the MER TKI can be used to ramp up the immune response to the tumor by inhibiting macrophage tumorogenic tolerance during normal tumor chemotherapeutic agent. The immunomodulatory dosage of the MER TKI can be given prior to, with or after chemotherapeutic therapy and can be used simultaneously with or intermittently with the chemotherapeutic therapy. In one embodiment, less chemotherapeutic therapy is needed than the normal standard of care defined for that chemotherapeutic agent, due to the increased efficacy of the immune response in the surrounding tumor microenvironment. Therefore, in one embodiment, a low dose of MER TKI (for example 1 to 100 mg/dose) is given as a type of adjunctive therapy with the chemotherapeutic agent.

In another embodiment, a tumor survival-signal inhibiting amount (for example at least 150 mg/dosage, and in some embodiments, at least 200, 250, 300, 350, 400, 450 or 500 mg/dosage or more) of MER TKI is administered to a host alone or in combination with a chemotherapeutic agent and/or anti-cancer targeted agent. In one aspect, the MER TKI and the chemotherapeutic agent act synergistically. In one embodiment, the use of a MER TKI in combination with a chemotherapeutic agent provides for increased anti-tumor effects without an increase in the standard of care dosage of the chemotherapeutic agent.

In one embodiment, the use of a MER TKI in combination with a chemotherapeutic provides for equivalent or increased anti-tumor effects utilizing a lower dosage of a chemotherapeutic agent than the standard of care dosage. In one embodiment, the cancer is a solid tumor. In one embodiment, the cancer is selected from a leukemia, lymphoma, lung cancer, melanoma, breast, pancreatic, and glioblastoma. In one embodiment, the cancer is Acute Lymphoblastic Leukemia (ALL). In one embodiment, the cancer is Acute Myeloid Leukemia (AML). In one embodiment, the cancer is ALL or AML and the chemotherapeutic is methotrexate.

In one aspect of the invention, the MER TKI can be administered to a host with a cancer prior to, during, or after administration with a chemotherapeutic agent or exposure to ionizing radiation. In one embodiment, a host is administered an effective amount of a chemotherapeutic agent or ionizing radiation and subsequently administered a MER TKI. In one embodiment, the MER TKI is administered as immunomodulatory agent. Without wanting to be bound by any particular theory, it is believed that the administration of a chemotherapeutic agent results in the apoptosis of tumor cells, exposing antigenic tumor proteins. The host's innate immune system is thus stimulated to recognize the antigenic apoptotic components from the tumor cells after chemotherapy or ionizing radiation and mount an immune response. In one embodiment, the administration of a chemotherapeutic agent or ionizing radiation, before, with or subsequently followed by the administration of a MER TKI is carried out using the normal standard of care chemotherapeutic protocol. In another embodiment, the standard of care protocol of the chemotherapeutic is changed in a manner that causes less toxicity to the host due to the adjunctive or synergistic activity of the MER TKI.

In one embodiment, the dose associated with the immunomodulatory effect is about 2-fold, about 3-fold, about 4-fold, about 5-fold, about 6-fold, about 7-fold, about 8-fold, about 9-fold, about 10-fold or greater lower than the dose associated with a direct survival-signal inhibiting anti-tumor or cytotoxic effect. In one embodiment, the dose used to induce an immunomodulatory effect in a host is between about 0.5 mg and about 150 mg. In one embodiment, the dose is about 1 mg, about 2 mg, about 3 mg, about 4 mg, about 5 mg, about 10 mg, about 12 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 125 mg, about 140 mg, or about 150 mg. In one embodiment, the tumor is a solid cancer. In one embodiment, the cancer is a MER (−/−) cancer. In one embodiment, the cancer is a MER (−/−) breast cancer. In one embodiment, the cancer is selected from the group consisting of lung, melanoma, breast, leukemia, and glioblastoma.

In one aspect of the invention, a method is provided to treat a host having a cancer by administering a once daily, oral tumor survival-signal inhibiting amount of a MER TKI. In one embodiment, the MER TKI dose is between about 200 mg and 1250 mg. In one embodiment, the dose is about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg or more. In one embodiment, the cancer is a solid tumor. In one embodiment, the cancer is a leukemia. In one embodiment, the leukemia is ALL. In one embodiment, the leukemia is AML. In one embodiment, the cancer is NSCLC. In one embodiment, the cancer is a melanoma. In one embodiment, the cancer is breast cancer. In one embodiment, the cancer is a glioblastoma.

In one aspect of the invention, a method is provided to treat a host having melanoma by administering to the host an effective amount of a MER TKI. In one embodiment, the administration of the MER TKI is combined with a chemotherapeutic agent. In one embodiment, the chemotherapeutic agent is an anti-programmed cell death −1 (PD-1) agent. In one embodiment, the chemotherapeutic agent is a B-RAF inhibitor. In one embodiment, the B-RAF inhibitor is vemurafenib. In one embodiment, the host does not have a melanoma with a B-RAF mutation. In one embodiment, the host has a melanoma with a B-RAF mutation. In one embodiment, the host has a melanoma with a RAS mutation. In one embodiment, the melanoma over-expresses MER. In one embodiment, the melanoma has metastasized.

In one aspect of the invention, a method is provided to treat a host with cancer comprising administering to the host an effective amount of a MER TKI in combination with an immunomodulatory agent. In one embodiment, the immunomodulatory agent is selected from the group consisting of a CTLA-4 inhibitor, PD-1 or anti-PD-1 ligand, IFN-alpha, IFN-beta, and a vaccine, for example, a cancer vaccine.

In one aspect of the invention, a method is provided to treat a host with cancer comprising administering to the host an effective amount of a MER TKI in combination with another anti-tyrosine kinase inhibitor. In one embodiment, the anti-tyrosine kinase inhibitor is a fibroblast growth factor receptor (FGFR) inhibitor. In one embodiment, the FGFR inhibitor is AZD-4547. In one embodiment, the cancer is non-small cell lung carcinoma (NSCLC).

In one embodiment, the MER TKIs useful in the present invention are dual MER/FLT-3 TKIs. In one embodiment, the MER TKIs are dual MER/Axl TKIs. In one embodiment, the MER TKIs are MER-specific TKIs.

### BRIEF DESCRIPTION OF FIGURES

FIG. 1A illustrates the Ig-like domain, FNIII domain and kinase domain in Tyro-3, Axl and Mer. FIG. 1B illustrates the Gla domain, EGF repeat, LG1 domain and LG2 domain in Mer TK. The Gas6/protein S loop region is also illustrated. FIG. 1C and FIG. 1D are scanning EM illustrates the binding of apoptotic thymocytes to Mer^{+/+} and Me^{-/-} macrophages. Wild-type macrophages ingest; Mer^{-/-} do not.

FIG. 2A illustrates the results obtained when MMTV-PVmT mammary tumors were implanted into MerTK^{+/-}, MerTK^{+/+} and MerTK^{-/-} mice. Tumors that were implanted into MerTK^{-/-} mice were almost 75% tumor free after 200 days. FIG. 2B illustrates the results when B16:F10 intradermal tumors were implanted in MerTK^{+/+} or MerTK^{-/-} mice. MerTK^{-/-} mice were tumor free for 40 days and MerTK^{+/+} mice were tumor free for approximately 28 days. FIG. 2C is a graph showing the number of lung metastases per mouse verses the genotype of mice transplanted with MMTV-PvVmT or B16:F10 tumor lines.

FIG. 3: Mer TK is a dual target in cancer. Mer TK is over expressed in tumor cells such as lung, melanoma and GBM and sends a survival signal. MerTK inhibitors inhibit tumor cell survival and chemoresistance. In addition, Mer TK is expressed in tumor-associated macrophages (e.g., breast, melanoma and lung cancer) and suppresses tumor immunity. Mer TK inhibitors stimulate innate anti-tumor immunity.

FIG. 4A and FIG. 4B demonstrate that UNC1666 abrogates Mer and Flt3 kinase phosphorylation and downstream signaling in leukemic blasts isolated from a patient with acute myeloid leukemia at concentrations of 50, 100 and 300 nM. FIGS. 4C and 4D illustrate the percent apoptotic and necrotic cells in when leukemic blasts isolated from patients with AMLs expressing Mer and/or a FLT3-ITD were treated with UNC1666 at concentrations of 50, 100 and 300 nM. FIGS. 4E and 4F are graphs of the relative number of colonies when AML cells isolated from patients were grown in the presence of UNC1666 at concentrations of 50, 100 and 300 nM.

FIG. 5A: MRX6313 is a potent Mer/FLT3 dual TK inhibitor. The compound has a Kᵢ=0.16 nM against Mer, a Kᵢ=0.71 nM against FLT3, a Kᵢ=15 nM against Axl and a Kᵢ=5.1 nM against Tyro3. Mice were dosed (iv and po) with 3 mg/kg MRX-6313. The graph illustrates the plasma concentration of MRX-6313 in ng/mL verses time in hours. FIG. 5B illustrates the pharmacokinetic parameters for MRX6313.

FIG. 6A and FIG. 6B show reduced tumor burden measured by bioluminescent imaging in response to treatment with MRX6313 relative to mice treated with vehicle in orthotopic B-ALL xenograft models of established disease (A) and minimal residual disease (B). FIG. 6C shows mean bioluminescence intensity verses day post-transplant in mice receiving saline qd or 100 mg/kg MRX6313 qd starting at day 12 post-transplant in the B-ALL xenograft model of established disease. FIG. 6D is a Kaplan Meier plot showing percent survival verses days post-transplant for mice receiving either saline or 100 mg/kg MRX6313 in the B-ALL xenograft model of established disease. FIG. 6E shows mean bioluminescence intensity verses day post-transplant in mice receiving saline qd or 75 mg/kg MRX6313 qd starting at day 1 in the B-ALL xenograft model of minimal residual disease.

FIG. 6F is a Kaplan Meier plot showing percent survival verses days post-transplant for mice receiving either saline or 75 mg/kg MRX6313 in the B-ALL xenograft model of minimal residual disease. FIGS. 6G-6H show median survival for mice receiving saline, 100 mg/kg MRX6313 qd starting at day 12, or 75 mg/kg MRX6313 qd starting at day 1.

FIG. 7A is a graph illustrating the average bioluminescence intensity (×10⁶ photons/sec) verses days post-transplant in mice treated with 75 mg/kg MRX6313, 1 mg/kg methotrexate (MTX) or 75 mg/kg MRX6313+1 mg/kg methotrexate in a B-ALL xenograft model. FIG. 7B is a graph showing tumor burden 88 days post-transplant in individual mice treated with 75 mg/kg MRX6313+1 mg/kg methotrexate in the experiment shown in 7A. FIG. 7C is Kaplan Meier plot illustrating % leukemia-free survival verses days post-transplant for mice treated with 75 mg/kg MRX6313, 1 mg/kg methotrexate (MTX) or 75 mg/kg MRX6313+1 mg/kg methotrexate in the B-ALL xenograft model. FIG. 7D is a table illustrating median survival when mice were dosed with 75 mg/kg MRX-6313 QD starting at d12, 1 mg/kg MTX QD×2 d/wk×7 cycles starting at d14, or 75 mg/kg MRX6313 d12 and 1 mg/kg MTX d14 (n=5).

FIG. 8A shows reduced tumor volume in response to treatment with 50 mg/kg MRX6313 in a subcutaneous xenograft model of NSCLC established using Mer+, FGFR+Colo699 cells. FIG. 8B: H226 (Mer+, FGFR+) NSCLC cells were cultured for 14 days in soft agar in the presence of MRX6313 and/or AZD-4547, alone or in combination, and colonies were stained and counted.

FIGS. 9A, 9B and 9C show Mer protein expression detected by immunocytochemistry in melanocytes (S100-positive) in nevus (A), primary melanoma (B), and metastatic melanoma (C) samples. FIG. 9D is an immunoblot that shows that MRX6313 abrogates signaling downstream of MER in the melanoma cell lines HMCB and G361. FIG. 9E is a graph illustrating relative colony number when HMCB and G361 cells were dosed with MRX6313 at 25, 50, 100, 300 and 500 nM.

FIG. 10A is an immunoblot showing the presence of Mer TK in J774 murine macrophage and absence in PyVmT murine mammary tumor cells. FIGS. 10B and 10C illustrate the results from an immune-competent orthotopic model of Mer-negative breast cancer. Mice were transplanted with PyVmT mammary tumor cells and were treated with 50 mg/kg MRX6313 or vehicle qd starting 2 days before transplant. FIG. 10B is a graph showing tumor volume (mm³) verses days post-tumor injection. FIG. 10C illustrates proinflammatory signaling pathway components that exhibit altered expression in tumor-associated macrophages following treatment with MRX6313 determined by RNA sequencing.

FIG. 11 is a graph illustrating percent change in melanoma tumor volume in a genetically-engineered mouse (GEM) model (TRIA) after 21 days of treatment with various drugs and combinations of drugs. MEK plus P13K (AZD6244/BEZ235) was the only regimen to show efficacy in the model. This combination is not tolerated in humans.

FIG. 12A is a graph illustrating tumor volume (mm³) verses days when in TRIA mice treated with MRX6313 or vehicle. FIG. 12B is a Kaplan Meier plot showing percent survival verses days on MRX6313 therapy in the TRIA GEM model. FIG. 12C is a Kaplan Meier plot of percent survival verses days on therapy verses no treatment in the TRIA GEM model. Mice with melanoma were treated with MRX6313, AZD6244/BEZ235 or given no treatment.

FIG. 13: Mer TK is a dual target in cancer. Mer TK is over-expressed in tumor cells such as leukemia, lung cancer, melanoma and GBM which results in a survival signal. Mer TK inhibitors reverse survival and chemoresistance. In addition, Mer TK is expressed in tumor macrophages, e.g., breast and lung cancer and suppresses tumor immunity. Mer TK inhibitors stimulate innate anti-tumor immunity.

FIG. 14 is a waterfall plot showing percent change in tumor volumes verses days of treatment in the TRIA GEM model of melanoma. Mice were treated with UNC2025, AZD6244, Carbo/Taxol or received no treatment. The best response was mediated by UNC2025.

FIG. 15 is a Kaplan Meier plot showing percent survival verses days on therapy in TRIA mice with melanoma treated with PD1, UNC2025, UNC2025 in combination with PD1 or received no treatment.

FIG. 16A is a graph illustrating percent change in tumor volume in TRIA mice with melanoma treated with UNC2025 or AZD6244 verses no treatment. AZD6244 is a MEK inhibitor that was administered in mouse chow at a predicted dose of 37 mg/kg (MTD). UNC2025 was administered in chow at a predicted dose of 120 mg/kg (MTD). FIG. 16B is a Kaplan Meier plot illustrating percent survival verses days of treatment when TRIA mice with melanoma were treated with no drug, UNC2025 or AZD6244. AZD6244 is a MEK inhibitor that was dosed in mouse chow at a predicted dose of 37 mg/kg (MTD). UNC2025 was dosed in chow at a predicted dose of 120 mg/kg (MTD).

FIG. 17A is a graph illustrating relative levels of Mer protein expression (AQUA score (log 2) determined by immunohistochemistry in nevus, primary melanoma, and metastatic melanoma samples. FIGS. 17B to 17F show reduced colony formation in soft agar in response to treatment with UNC1062 in B-RAF wild type HMCB (A) and B-RAF mutant G361 (B) melanoma cell lines. FIG. 17G shows reduced invasion into collagen matrix by SKMEL119 melanoma cells in response to treatment with UNC1062.

FIG. 18A is a graph illustrating tumor volume verses days treated. Colo699 tumor cells were subcutaneously implanted in mice. Mice subsequently received either saline or MRX6313, 50 mg/kg, qd. FIG. 18B is an immunoblot of pMer and downstream signaling proteins in the NSCLC cell line H1299 after 24 h pre-treatment with Axl siRNA and MRX6313 treatment for one hour.

FIG. 19A illustrates the number of colonies (H226) in a soft agar assay after two weeks of treatment. Colonies were treated with MRX6313 and AZD4547 at concentrations ranging from 0 to 100 nM. FIG. 19B is an immunoblot of signaling proteins downstream of Mer in the NSCLC cell line H226 after 4 h treatment with DMSO, AZD4547, MRX6313, or AZD4547 and MRX6313.

FIG. 20A is a graph of tumor volume (mm³) verses days. This is treatment of the intact GEMM. FIG. 20B is a graph illustrating the results from TRIA injected into NSG mice. The mice were then treated with no drug or UNC2025. Tumor volumes were significantly reduced when mice were treated with UNC2025 verses control. In FIGS. 20A and 20B, both treated slopes were statistically significant compared to untreated slopes using linear regression p<0.001. FIG. 20C; Pten/Braf genetically engineered mice were treated with no drug or UNC2025. The graph illustrates tumor volumes (mm³) verses days on treatment.

FIG. 21A is a timeline illustrating a mouse model using PyVmT cells. Mice were treated with 3 mg/kg MRX6313 or saline twice daily by oral gavage. Mice were dosed from day −2 to day 28. At day zero, 1×10⁶ PyVmT cells were implanted into the inguinal mammary fatpad of mice. Tumor volumes were measured at days 16, 19, 21, 23, 26 and 28. Day 28 was the end of the study. FIG. 21B is a graph illustrating tumor volume (mm) verses days post-tumor injection. RNA-sequence data indicate that MRX6313 treatment increases pro-inflammatory cytokines in CD11b⁺ cells and activates CD8⁺ T cell effector function.

FIG. 22A illustrates a Mer spleen verses a wild type spleen. FIG. 22B illustrates an enlarged Mer_{tg} lymph node. FIG. 22C is a picture of cells from a Mer_{tg} lymph node. FIG. 22D shows Mer expression in pediatric ALL patients detected by RT-PCR. FIGS. 22E and 22F illustrate the percent of adult and pediatric patients with acute myeloid leukemias that are Mer positive, Mer Dim, or Mer negative. See, Graham, Armistead et al., Oncogene, 2013.

FIG. 23 is the chemical structure of UNC1666.

FIG. 24 is the chemical structure of UNC2025/MRX6313.

FIG. 25A shows induction of apoptosis and cell death in cultures of the BRAF mutant G361 cell line treated with UNC1062, vemurafenib, or UNC1062 and vemurafenib. In addition, the percent of apoptotic and dead cells expected if the interaction between UNC1062 and vemurafenib is additive was calculated using the Bliss additivity model (Additive Fa). The percent apoptotic and dead cells observed (Actual Fa) was greater than the predicted additive value, indicating a synergistic interaction. FIG. 25B shows inhibition of signaling downstream of BRAF and/or Mer in response to treatment with UNC1062, vemurafenib, or UNC1062 and vemurafenib.

FIG. 26 shows the chemical structure of UNC1062.

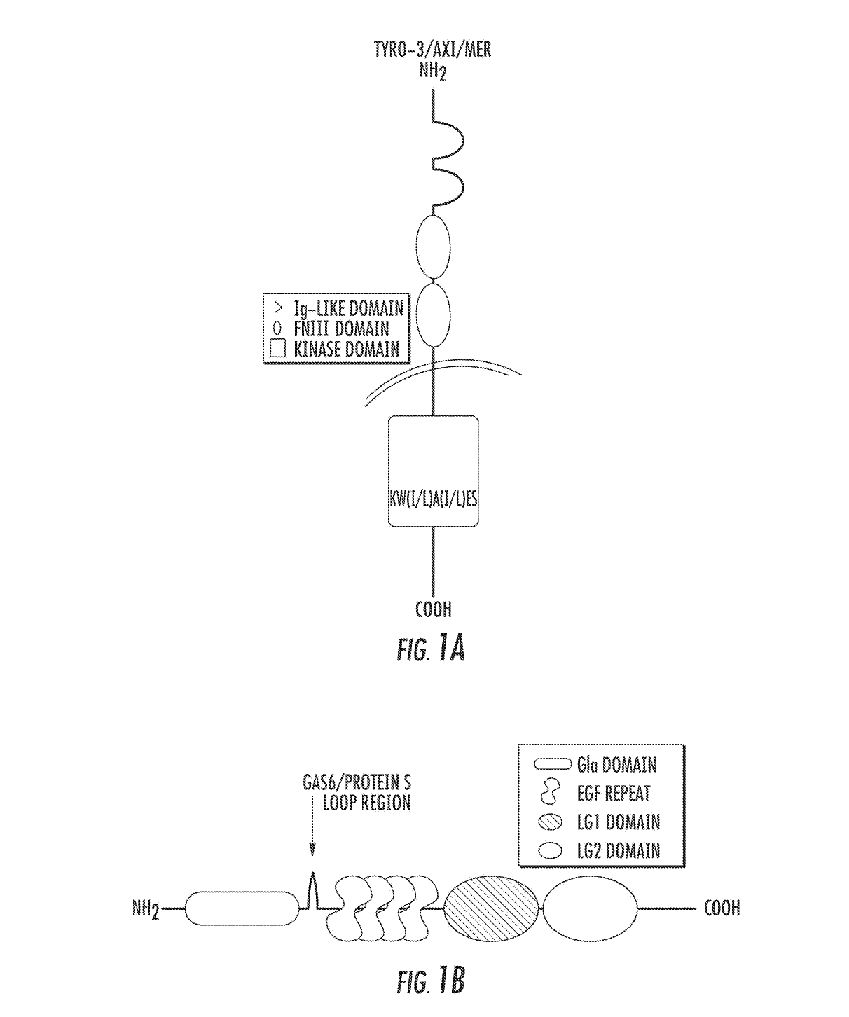

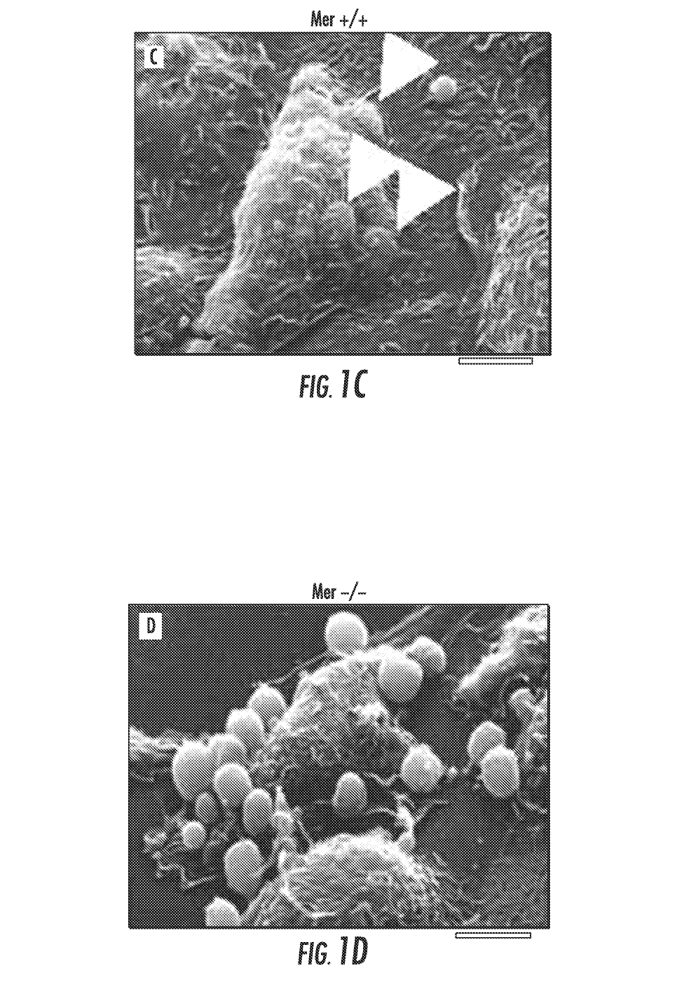

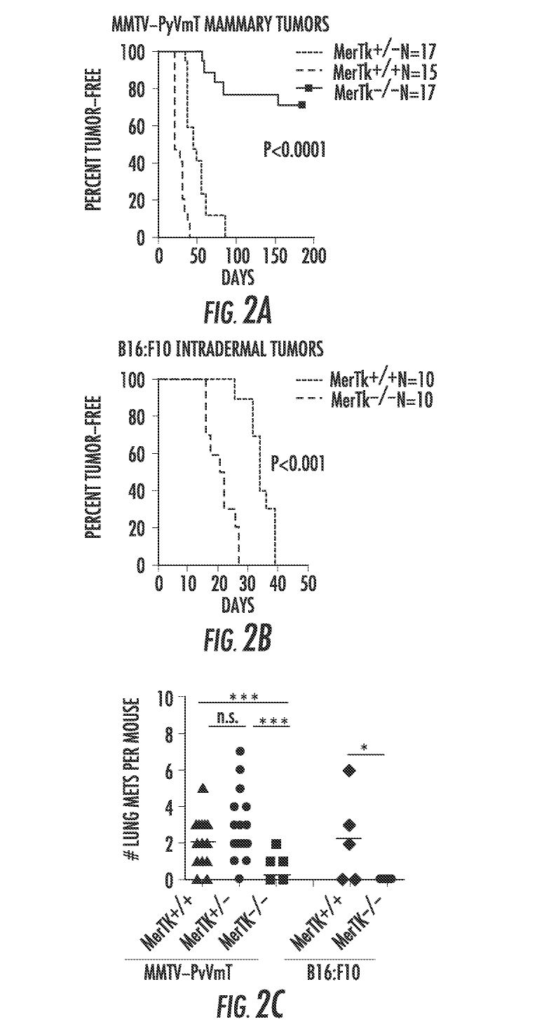

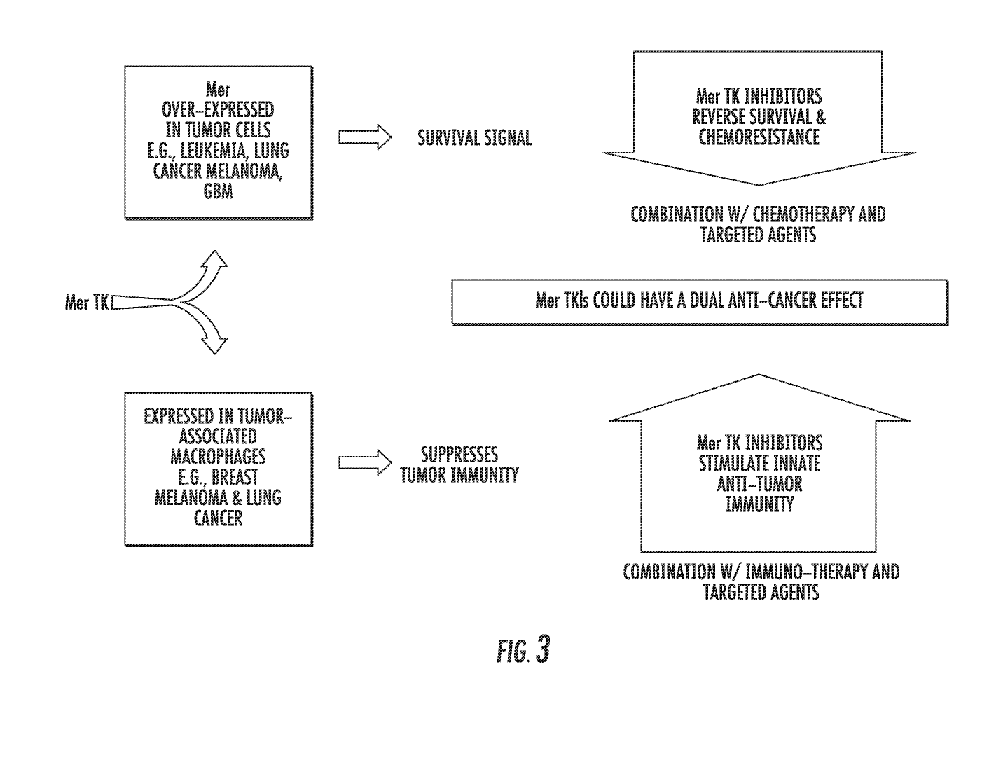

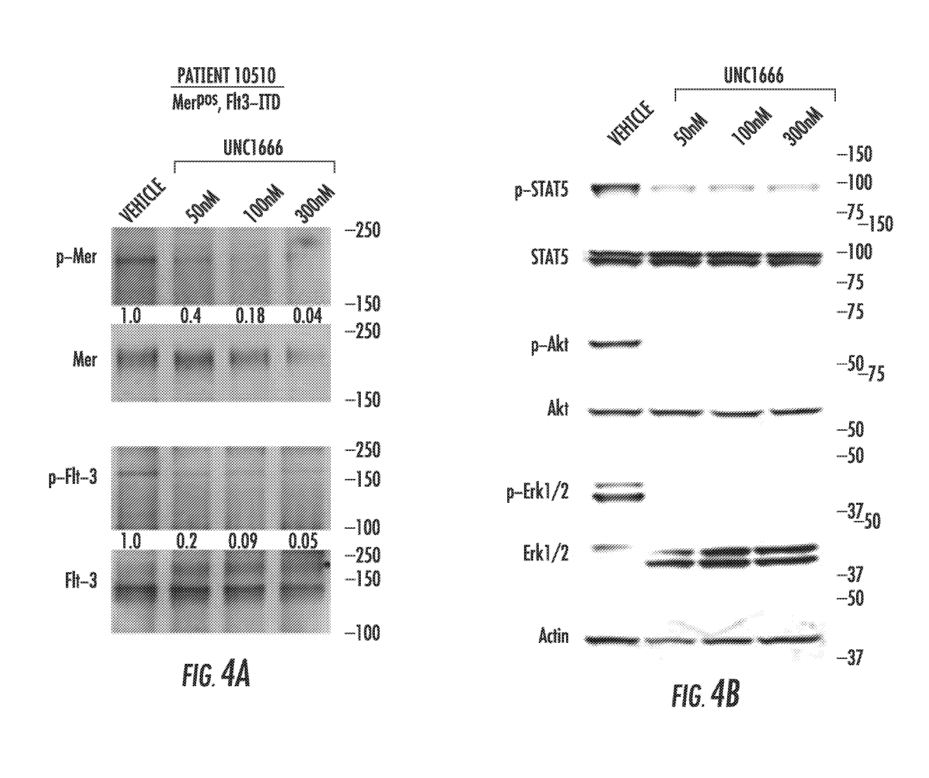

### DETAILED DESCRIPTION

The methods described herein are directed to the treatment of a host suffering from a tumor. The term “host” refers to an individual, typically a mammal such as a human. The term “host” can also include domesticated animals, such as cats, dogs, etc., livestock (e.g., cattle, horses, pigs, sheep, goats, etc.), laboratory animals (e.g., mouse, monkey, rabbit, rat, guinea pig, etc.) and birds.

MER Tyrosine Kinase Inhibitors

In addition to the specific MER TKIs described herein, the methods of the current invention can be utilized with MER TKIs described in WO2011146313, WO2013052417, WO2013177168, PCT/US2013/065192, and PCT/US2013/71409, incorporated herein in their entirety.

Tumors

The methods provided herein are useful for the treatment of tumors. As contemplated herein, the cancer treated can be a primary tumor or a metastatic tumor. In one aspect, the methods described herein are used to treat a solid tumor, for example, melanoma, lung cancer (including lung adenocarcinoma, basal cell carcinoma, squamous cell carcinoma, large cell carcinoma, bronchioloalveolar carcinoma, bronchiogenic carcinoma, non-small-cell carcinoma, small cell carcinoma, mesothelioma); breast cancer (including ductal carcinoma, lobular carcinoma, inflammatory breast cancer, clear cell carcinoma, mucinous carcinoma, serosal cavities breast carcinoma); colorectal cancer (colon cancer, rectal cancer, colorectal adenocarcinoma); anal cancer; pancreatic cancer (including pancreatic adenocarcinoma, islet cell carcinoma, neuroendocrine tumors); prostate cancer; prostate adenocarcinoma; ovarian carcinoma (ovarian epithelial carcinoma or surface epithelial-stromal tumor including serous tumor, endometrioid tumor and mucinous cystadenocarcinoma, sex-cord-stromal tumor); liver and bile duct carcinoma (including hepatocellular carcinoma, cholangiocarcinoma, hemangioma); esophageal carcinoma (including esophageal adenocarcinoma and squamous cell carcinoma); oral and oropharyngeal squamous cell carcinoma; salivary gland adenoid cystic carcinoma; bladder cancer; bladder carcinoma; carcinoma of the uterus (including endometrial adenocarcinoma, ocular, uterine papillary serous carcinoma, uterine clear-cell carcinoma, uterine sarcomas and leiomyosarcomas, mixed mullerian tumors); glioma, glioblastoma, medullablastoma, and other tumors of the brain; kidney cancers (including renal cell carcinoma, clear cell carcinoma, Wilm's tumor); cancer of the head and neck (including squamous cell carcinomas); cancer of the stomach (gastric cancers, stomach adenocarcinoma, gastrointestinal stromal tumor); testicular cancer; germ cell tumor; neuroendocrine tumor, cervical cancer; carcinoids of the gastrointestinal tract, breast, and other organs; signet ring cell carcinoma; mesenchymal tumors including sarcomas, fibrosarcomas, haemangioma, angiomatosis, haemangiopericytoma, pseudoangiomatous stromal hyperplasia, myofibroblastoma, fibromatosis, inflammatory myofibroblastic tumor, lipoma, angiolipoma, granular cell tumor, neurofibroma, schwannoma, angiosarcoma, liposarcoma, rhabdomyosarcoma, osteosarcoma, leiomyoma, leiomysarcoma, skin, including melanoma, cervical, retinoblastoma, head and neck cancer, pancreatic, brain, thyroid, testicular, renal, bladder, soft tissue, adenal gland, urethra, cancers of the penis, myxosarcoma, chondrosarcoma, osteosarcoma, chordoma, malignant fibrous histiocytoma, lymphangiosarcoma, mesothelioma, squamous cell carcinoma; epidermoid carcinoma, malignant skin adnexal tumors, adenocarcinoma, hepatoma, hepatocellular carcinoma, renal cell carcinoma, hypernephroma, cholangiocarcinoma, transitional cell carcinoma, choriocarcinoma, seminoma, embryonal cell carcinoma, glioma anaplastic; glioblastoma multiforme, neuroblastoma, medulloblastoma, malignant meningioma, malignant schwannoma, neurofibrosarcoma, parathyroid carcinoma, medullary carcinoma of thyroid, bronchial carcinoid, pheochromocytoma, Islet cell carcinoma, malignant carcinoid, malignant paraganglioma, melanoma, Merkel cell neoplasm, cystosarcoma phylloide, salivary cancers, thymic carcinomas, and cancers of the vagina among others.

In one embodiment, the methods described herein are useful for treating a host suffering from a lymphoma or lymphocytic or myelocytic proliferation disorder or abnormality. For example, the MER TKIs as described herein can be administered to a subject suffering from a Hodgkin Lymphoma of a Non-Hodgkin Lymphoma. For example, the subject can be suffering from a Non-Hodgkin Lymphoma such as, but not limited to: an AIDS-Related Lymphoma; Anaplastic Large-Cell Lymphoma; Angioimmunoblastic Lymphoma; Blastic NK-Cell Lymphoma; Burkitt's Lymphoma; Burkitt-like Lymphoma (Small Non-Cleaved Cell Lymphoma); Chronic Lymphocytic Leukemia/Small Lymphocytic Lymphoma; Cutaneous T-Cell Lymphoma; Diffuse Large B-Cell Lymphoma; Enteropathy-Type T-Cell Lymphoma; Follicular Lymphoma; Hepatosplenic Gamma-Delta T-Cell Lymphoma; Lymphoblastic Lymphoma; Mantle Cell Lymphoma; Marginal Zone Lymphoma; Nasal T-Cell Lymphoma; Pediatric Lymphoma; Peripheral T-Cell Lymphomas; Primary Central Nervous System Lymphoma; T-Cell Leukemias; Transformed Lymphomas; Treatment-Related T-Cell Lymphomas; or Waldenstrom's Macroglobulinemia.

Alternatively, the subject may be suffering from a Hodgkin Lymphoma, such as, but not limited to: Nodular Sclerosis Classical Hodgkin's Lymphoma (CHL); Mixed Cellularity CHL; Lymphocyte-depletion CHL; Lymphocyte-rich CHL; Lymphocyte Predominant Hodgkin Lymphoma; or Nodular Lymphocyte Predominant HL.

In one embodiment, the methods as described herein may be useful to treat a host suffering from a specific T-cell, a B-cell, or a NK-cell based lymphoma, proliferative disorder, or abnormality. For example, the subject can be suffering from a specific T-cell or NK-cell lymphoma, for example, but not limited to: Peripheral T-cell lymphoma, for example, peripheral T-cell lymphoma and peripheral T-cell lymphoma not otherwise specified (PTCL-NOS); anaplastic large cell lymphoma, for example anaplastic lymphoma kinase (ALK) positive, ALK negative anaplastic large cell lymphoma, or primary cutaneous anaplastic large cell lymphoma; angioimmunoblastic lymphoma; cutaneous T-cell lymphoma, for example mycosis fungoides, Sézary syndrome, primary cutaneous anaplastic large cell lymphoma, primary cutaneous CD30+ T-cell lymphoproliferative disorder, primary cutaneous aggressive epidermotropic CD8+ cytotoxic T-cell lymphoma; primary cutaneous gamma-delta T-cell lymphoma; primary cutaneous small/medium CD4+ T-cell lymphoma. and lymphomatoid papulosis; Adult T-cell Leukemia/Lymphoma (ATLL); Blastic NK-cell Lymphoma; Enteropathy-type T-cell lymphoma; Hematosplenic gamma-delta T-cell Lymphoma; Lymphoblastic Lymphoma; Nasal NK/T-cell Lymphomas; Treatment-related T-cell lymphomas; for example lymphomas that appear after solid organ or bone marrow transplantation; T-cell prolymphocytic leukemia; T-cell large granular lymphocytic leukemia; Chronic lymphoproliferative disorder of NK-cells; Aggressive NK cell leukemia; Systemic EBV+ T-cell lymphoproliferative disease of childhood (associated with chronic active EBV infection); Hydroa vacciniforme-like lymphoma; Adult T-cell leukemia/lymphoma; Enteropathy-associated T-cell lymphoma; Hepatosplenic T-cell lymphoma; or Subcutaneous panniculitis-like T-cell lymphoma.

Alternatively, the subject may be suffering from a specific B-cell lymphoma or proliferative disorder such as, but not limited to: multiple myeloma; Diffuse large B cell lymphoma; Follicular lymphoma; Mucosa-Associated Lymphatic Tissue lymphoma (MALT); Small cell lymphocytic lymphoma; Mantle cell lymphoma (MCL); Burkitt lymphoma; Mediastinal large B cell lymphoma; Waldenström macroglobulinemia; Nodal marginal zone B cell lymphoma (NMZL); Splenic marginal zone lymphoma (SMZL); Intravascular large B-cell lymphoma; Primary effusion lymphoma; or Lymphomatoid granulomatosis; Chronic lymphocytic leukemia/small lymphocytic lymphoma; B-cell prolymphocytic leukemia; Hairy cell leukemia; Splenic lymphoma/leukemia, unclassifiable; Splenic diffuse red pulp small B-cell lymphoma; Hairy cell leukemia-variant; Lymphoplasmacytic lymphoma; Heavy chain diseases, for example, Alpha heavy chain disease, Gamma heavy chain disease, Mu heavy chain disease; Plasma cell myeloma; Solitary plasmacytoma of bone; Extraosseous plasmacytoma; Primary cutaneous follicle center lymphoma; T cell/histiocyte rich large B-cell lymphoma; DLBCL associated with chronic inflammation; Epstein-Barr virus (EBV)+ DLBCL of the elderly; Primary mediastinal (thymic) large B-cell lymphoma; Primary cutaneous DLBCL, leg type; ALK+ large B-cell lymphoma; Plasmablastic lymphoma; Large B-cell lymphoma arising in HHV8-associated multicentric; Castleman disease; B-cell lymphoma, unclassifiable, with features intermediate between diffuse large B-cell lymphoma and Burkitt lymphoma; B-cell lymphoma, unclassifiable, with features intermediate between diffuse large B-cell lymphoma and classical Hodgkin lymphoma; Nodular sclerosis classical Hodgkin lymphoma; Lymphocyte-rich classical Hodgkin lymphoma; Mixed cellularity classical Hodgkin lymphoma; or Lymphocyte-depleted classical Hodgkin lymphoma.

In one embodiment, the methods described herein can be used to a subject suffering from a leukemia. For example, the subject may be suffering from an acute or chronic leukemia of a lymphocytic or myelogenous origin, such as, but not limited to: Acute lymphoblastic leukemia (ALL); Acute myelogenous leukemia (AML); Chronic lymphocytic leukemia (CLL); Chronic myelogenous leukemia (CML); juvenile myelomonocytic leukemia (JMML); hairy cell leukemia (HCL); acute promyelocytic leukemia (a subtype of AML); T-cell prolymphocytic leukemia (TPLL); large granular lymphocytic leukemia; or Adult T-cell chronic leukemia; large granular lymphocytic leukemia (LGL). In one embodiment, the patient suffers from an acute myelogenous leukemia, for example an undifferentiated AML (M0); myeloblastic leukemia (M1; with/without minimal cell maturation); myeloblastic leukemia (M2; with cell maturation); promyelocytic leukemia (M3 or M3 variant [M3V]); myelomonocytic leukemia (M4 or M4 variant with eosinophilia [M4E]); monocytic leukemia (M5); erythroleukemia (M6); or megakaryoblastic leukemia (M7).

Chemotherapeutic Agents

In one embodiment, a MER TKI is used in combination with a chemotherapeutic agent. Such agents may include, but are not limited to, tamoxifen, midazolam, letrozole, bortezomib, anastrozole, goserelin, an mTOR inhibitor, a PI3 kinase inhibitors, dual mTOR-PI3K inhibitors, MEK inhibitors, RAS inhibitors, ALK inhibitors, HSP inhibitors (for example, HSP70 and HSP 90 inhibitors, or a combination thereof). Examples of mTOR inhibitors include but are not limited to rapamycin and its analogs, everolimus (Afinitor), temsirolimus, ridaforolimus, sirolimus, and deforolimus. Examples of P13 kinase inhibitors include but are not limited to Wortmannin, demethoxyviridin, perifosine, idelalisib, PX-866, IPI-145, BAY 80-6946, BEZ235, RP6503, TGR 1202 (RP5264), MLN1117 (INK117), Pictilisib, Buparlisib, SAR245408 (XL147), SAR245409 (XL765), Palomid 529, ZSTK474, PWT33597, RP6530, CUDC-907, and AEZS-136. Examples of MEK inhibitors include but are not limited to Tametinib, Selumetinib, MEK162, GDC-0973 (XL518), and PD0325901. Examples of RAS inhibitors include but are not limited to Reolysin and siG12D LODER. Examples of ALK inhibitors include but are not limited to Crizotinib, AP26113, and LDK378. HSP inhibitors include but are not limited to Geldanamycin or 17-N-Allylamino-17-demethoxygcldanamycin (17AAG), and Radicicol. In one embodiment, the chemotherapeutic agent is an anti-programmed cell death −1 (PD-1) agent, for example, nivolumab, BMS936559, lambrolizumab, MPDL3280A, pidilizumab, In one embodiment, the chemotherapeutic agent is a B-RAF inhibitor, for example, vemurafenib or sorafenib. In one embodiment, the chemotherapeutic agent is a FGFR inhibitor, for example, but not limited to, AZD4547, dovitinib, BGJ398, LY2874455, and ponatinib.

Other chemotherapeutic agents that can be used in combination with the compounds described herein include, but are not limited to, chemotherapeutic agents that do not require cell cycle activity for their anti-neoplastic effect.

In certain aspects, the additional therapeutic agent is an anti-inflammatory agent, a chemotherapeutic agent, a radiotherapeutic, additional therapeutic agents, or immunosuppressive agents.

Suitable chemotherapeutic agents include, but are not limited to, radioactive molecules, toxins, also referred to as cytotoxins or cytotoxic agents, which includes any agent that is detrimental to the viability of cells, agents, and liposomes or other vesicles containing chemotherapeutic compounds. General anticancer pharmaceutical agents include: Vincristine (Oncovin®) or liposomal vincristine (Marqibo®), Daunorubicin (daunomycin or Cerubidine®) or doxorubicin (Adriamycin®), Cytarabine (cytosine arabinoside, ara-C, or Cytosar®), L-asparaginase (Elspar®) or PEG-L-asparaginase (pegaspargase or Oncaspar®), Etoposide (VP-16), Teniposide (Vumon®), 6-mercaptopurine (6-MP or Purinethol®), Methotrexate, Cyclophosphamide (Cytoxan®), Prednisone, Dexamethasone (Decadron), imatinib (Gleevec®), dasatinib (Sprycel®), nilotinib (Tasigna®), bosutinib (Bosulif®), and ponatinib (Iclusig™). Examples of additional suitable chemotherapeutic agents include but are not limited to 1-dehydrotestosterone, 5-fluorouracil decarbazine, 6-mercaptopurine, 6-thioguanine, actinomycin D, adriamycin, aldesleukin, alkylating agents, allopurinol sodium, altretamine, amifostine, anastrozole, anthramycin (AMC)), anti-mitotic agents, cis-dichlorodiamine platinum (II) (DDP) cisplatin), diamino dichloro platinum, anthracyclines, antibiotics, antimetabolites, asparaginase, BCG live (intravesical), betamethasone sodium phosphate and betamethasone acetate, bicalutamide, bleomycin sulfate, busulfan, calcium leucouorin, calicheamicin, capecitabine, carboplatin, lomustine (CCNU), carmustine (BSNU), Chlorambucil, Cisplatin, Cladribine, Colchicin, conjugated estrogens, Cyclophosphamide, Cyclothosphamide, Cytarabine, Cytarabine, cytochalasin B, Cytoxan, Dacarbazine, Dactinomycin, dactinomycin (formerly actinomycin), daunirubicin HCL, daunorucbicin citrate, denileukin diftitox, Dexrazoxane, Dibromomannitol, dihydroxy anthracin dione, Docetaxel, dolasetron mesylate, doxorubicin HCL, dronabinol, E. coli L-asparaginase, emetine, epoetin-α, Erwinia L-asparaginase, esterified estrogens, estradiol, estramustine phosphate sodium, ethidium bromide, ethinyl estradiol, etidronate, etoposide citrororum factor, etoposide phosphate, filgrastim, floxuridine, fluconazole, fludarabine phosphate, fluorouracil, flutamide, folinic acid, gemcitabine HCL, glucocorticoids, goserelin acetate, gramicidin D, granisetron HCL, hydroxyurea, idarubicin HCL, ifosfamide, interferon α-2b, irinotecan HCL, letrozole, leucovorin calcium, leuprolide acetate, levamisole HCL, lidocaine, lomustine, maytansinoid, mechlorethamine HCL, medroxyprogesterone acetate, megestrol acetate, melphalan HCL, mercaptipurine, mesna, methotrexate, methyltestosterone, mithramycin, mitomycin C, mitotane, mitoxantrone, nilutamide, octreotide acetate, ondansetron HCL, paclitaxel, pamidronate disodium, pentostatin, pilocarpine HCL, plimycin, polifeprosan 20 with carmustine implant, porfimer sodium, procaine, procarbazine HCL, propranolol, rituximab, sargramostim, streptozotocin, tamoxifen, taxol, teniposide, tenoposide, testolactone, tetracaine, thioepa chlorambucil, thioguanine, thiotepa, topotecan HCL, toremifene citrate, trastuzumab, tretinoin, valrubicin, vinblastine sulfate, vincristine sulfate, and vinorelbine tartrate.

Additional therapeutic agents that can be administered in combination with a compound disclosed herein can include bevacizumab, sutinib, sorafenib, 2-methoxyestradiol or 2ME2, finasunate, vatalanib, vandetanib, aflibercept, volociximab, etaracizumab (MEDI-522), cilengitide, erlotinib, cetuximab, panitumumab, gefitinib, trastuzumab, dovitinib, figitumumab, atacicept, rituximab, alemtuzumab, aldesleukine, atlizumab, tocilizumab, temsirolimus, everolimus, lucatumumab, dacetuzumab, HLL1, huN901-DM1, atiprimod, natalizumab, bortezomib, carfilzomib, marizomib, tanespimycin, saquinavir mesylate, ritonavir, nelfinavir mesylate, indinavir sulfate, belinostat, panobinostat, mapatumumab, lexatumumab, dulanermin, ABT-737, oblimersen, plitidepsin, talmapimod, P276-00, enzastaurin, tipifarnib, perifosine, imatinib, dasatinib, lenalidomide, thalidomide, simvastatin, ABT-888, temozolomide, erlotinib, lapatinib, sunitinib, FTS, AZD6244, BEZ235, and celecoxib.

Immunomodulatory Agents

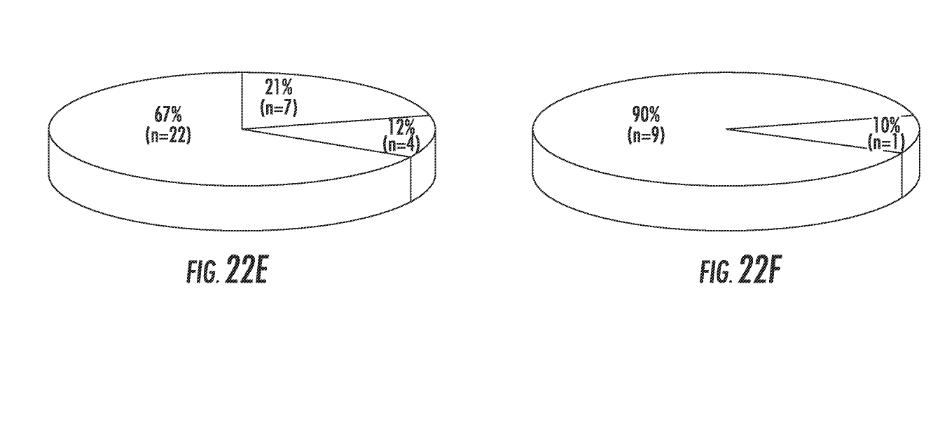

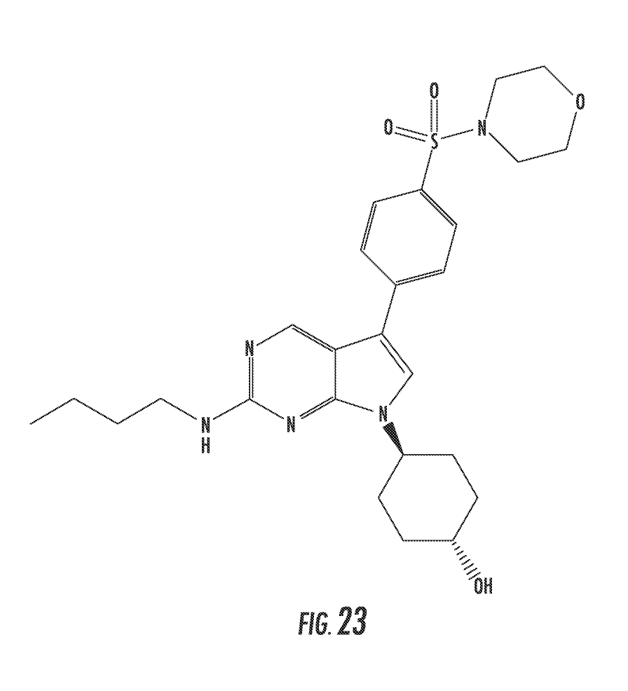

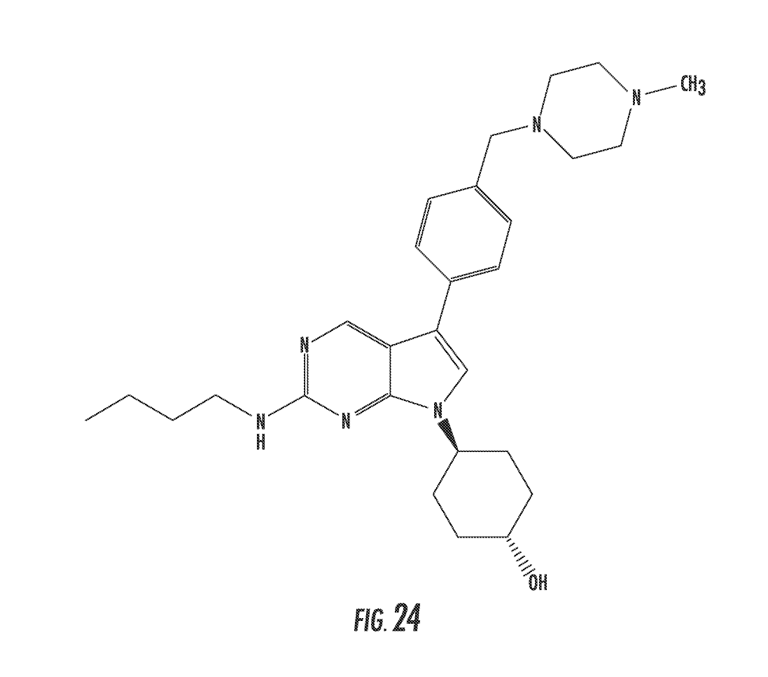

Mer tyrosine kinase inhibitors, including those described in FIGS. 23 and 24, and WO2011146313, WO2013052417, WO2013177168, PCT/US2013/065192, and PCT/US2013/71409, can be used in combination with one or more immunotherapy agents for additive or synergistic efficacy against solid tumors. In one embodiment, a tumor associated macrophage MER TK inhibiting amount of a MER TKI is used in combination or alternation with the immunomodulatory agent. In another embodiment, a host tumor survival-signal inhibiting amount of a MER TKI is used in combination or alternation with the immunomodulatory agent.

Immunomodulators are small molecules or biologic agents that treat a disease by inducing, enhancing or suppressing the host's immune system. In the present application, one or more immunomodulators are selected that induce or enhance the host's immune system. Some immunomodulators boost the host's immune system and others help train the host's immune system to better attack tumor cells. Other immunomodulators target proteins that help cancer grow.

Three general categories of immunotherapies are antibodies, cancer vaccines, and non-specific immunotherapies. Antibodies are typically administered as monoclonals, although that is not required. “Naked monoclonal antibodies” work by attaching to antigens on tumor cells. Some antibodies can act as a marker for the body's immune system to destroy the tumor cells. Others block signaling agents for tumor cells. Antibodies can generally be used to bind to any signaling or metabolic agent that directly or indirectly facilitates tumor growth. Examples are alemtuzumab (Campath) which binds to CD52 antigen, and trastuzumab (Herceptin), which binds to the HER2 protein.

In another embodiment, an antibody can be used that is conjugated to another moiety that increases it delivery or efficacy. For example, the antibody can be connected to a cytotoxic drug or a radiolabel. Conjugated antibodies are sometimes referred to as “tagged, labeled or loaded”. Radiolabeled antibodies have small radioactive particles attached to them. Examples are Zevalin, which is an antibody against CD20 used to treat lymphoma. Chemolabeled antibodies are antibodies that have cytotoxic agents attached to them. Examples are Adcetris, which targets CD30, and Kadcyla, which targets HER2. Ontak, while not an antibody, is similar in that it is interleukin-2 attached to a toxin from diphtheria.

Another category of immunotherapy that can be used in the present invention is a cancer vaccine. Most cancer vaccines are prepared from tumor cells, parts of tumor cells or pure antigens. The vaccine can be used with an adjuvant to help boost the immune response. An example is Provenge, which is the first cancer vaccine approved by the US FDA. The vaccine can for example be a dendritic cell vaccine or a vector-based vaccine

Nonspecific tumor immunotherapies and adjuvants include compounds that stimulate the immune system to do a better job at attacking the tumor cells. Such immunotherapies include cytokines, interleukins, interferons (α primarily but can be also β or γ). Specific agents include granulocyte-macrophage colony-stimulating factor (GM-CSF), IL-12, IL-7, IL-21, drugs that target CTLA-4 (such as Yervoy, which is Ipilimumab) and drugs that target PD-1 or PDL-1 (such as nivolumab or lambrolizumab).

Other drugs that boost the immune system are thalidomide, lenalidomide, pomalidomide, the Bacille Calmette-Gurin bacteria and Imiquimod. Additional therapeutic agents that can be used in combination with the Mer inhibitor include bispecific antibodies, chimeric antigen receptor (CAR) T-cell therapy and tumor-infiltrating lymphocytes.

Other immunomodulatory agents useful in combination therapies with MER TKIs as described herein include, but are not limited to, In one aspect of the present invention, a compound described herein can be combined with at least one immunosuppressive agent. The immunosuppressive agent is preferably selected from the group consisting of a calcineurin inhibitor, e.g. a cyclosporin or an ascomycin, e.g. Cyclosporin A (NEORAL®), FK506 (tacrolimus), pimecrolimus, a mTOR inhibitor, e.g. rapamycin or a derivative thereof, e.g. Sirolimus (RAPAMUNE®), Everolimus (Certican®), temsirolimus, zotarolimus, biolimus-7, biolimus-9, a rapalog, e.g. ridaforolimus, azathioprine, campath 1H, a SIP receptor modulator, e.g. fingolimod or an analogue thereof, an anti IL-8 antibody, mycophenolic acid or a salt thereof, e.g. sodium salt, or a prodrug thereof, e.g. Mycophenolate Mofetil (CELLCEPT®), OKT3 (ORTHOCLONE OKT3®), Prednisone, ATGAM®, THYMOGLOBULIN®, Brequinar Sodium, OKT4, T10B9.A-3A, 33B3.1, 15-deoxyspergualin, tresperimus, Leflunomide ARAVA®, CTLAI-Ig, anti-CD25, anti-IL2R, Basiliximab (SIMULECT®), Daclizumab (ZENAPAX®), mizorbine, methotrexate, dexamethasone, ISAtx-247, SDZ ASM 981 (pimecrolimus, Elidel®), CTLA41g (Abatacept), belatacept, LFA31g, etanercept (sold as Enbrel® by Immunex), adalimumab (Humira®), infliximab (Remicade®), an anti-LFA-1 antibody, natalizumab (Antegren®), Enlimomab, gavilimomab, antithymocyte immunoglobulin, siplizumab, Alefacept efalizumab, pentasa, mesalazine, asacol, codeine phosphate, benorylate, fenbufen, naprosyn, diclofenac, etodolac and indomethacin, aspirin and ibuprofen.

Pharmaceutical Compositions and Dosage Forms

In one aspect, the invention provides a pharmaceutical composition comprising a pharmaceutically effective amount of a MER TKI compound of the present invention and a pharmaceutically acceptable carrier.

The compounds provided herein are administered for medical therapy in a therapeutically effective amount. The amount of the compounds administered will typically be determined by a physician, in the light of the relevant circumstances, including the condition to be treated, the chosen route of administration, the compound administered, the age, weight, and response of the individual patient, the severity of the patient's symptoms, and the like.

The pharmaceutical compositions provided herein can be administered by a variety of routes including oral, parenteral, topical, rectal, subcutaneous, intravenous, intramuscular, and intranasal with a pharmaceutical carrier suitable for such administration. In one embodiment, the compounds are administered in a controlled release formulation.

The compositions for oral administration can take the form of bulk liquid solutions or suspensions, or bulk powders. Typically, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (as a nonlimiting example, from about 0.1 to about 50% by weight or preferably from about 1 to about 40% by weight) with the remainder being various vehicles or carriers and processing aids helpful for forming the desired dosing form. In one embodiment, the compound is present from about 1% to about 10% by weight.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art.

The above-described components for orally administrable or injectable compositions are merely representative. Other materials as well as processing techniques and the like are set forth in Part 8 of Remington's Pharmaceutical Sciences, 17th edition, 1985, Mack Publishing Company, Easton, Pa., which is incorporated herein by reference.

The MER TKI compound of this invention can also be administered in sustained release forms or from sustained release drug delivery systems. A description of representative sustained release materials can be found in Remington's Pharmaceutical Sciences.

In certain embodiments, the formulation comprises water. In another embodiment, the formulation comprises a cyclodextrin derivative. In certain embodiments, the formulation comprises hexapropyl-β-cyclodextrin. In a more particular embodiment, the formulation comprises hexapropyl-β-cyclodextrin (10-50% in water).

The present invention also includes pharmaceutically acceptable acid addition salts of compounds of the compounds of the invention. The acids which are used to prepare the pharmaceutically acceptable salts are those which form non-toxic acid addition salts, i.e. salts containing pharmacologically acceptable anions such as the hydrochloride, hydroiodide, hydrobromide, nitrate, sulfate, bisulfate, phosphate, acetate, lactate, citrate, tartrate, succinate, maleate, fumarate, benzoate, para-toluenesulfonate, and the like.

### EXAMPLES

### Example 1

Efficacy of a novel small molecule MER receptor tyrosine kinase inhibitor in B-RAF wild-type and B-RAF mutant melanoma cell.

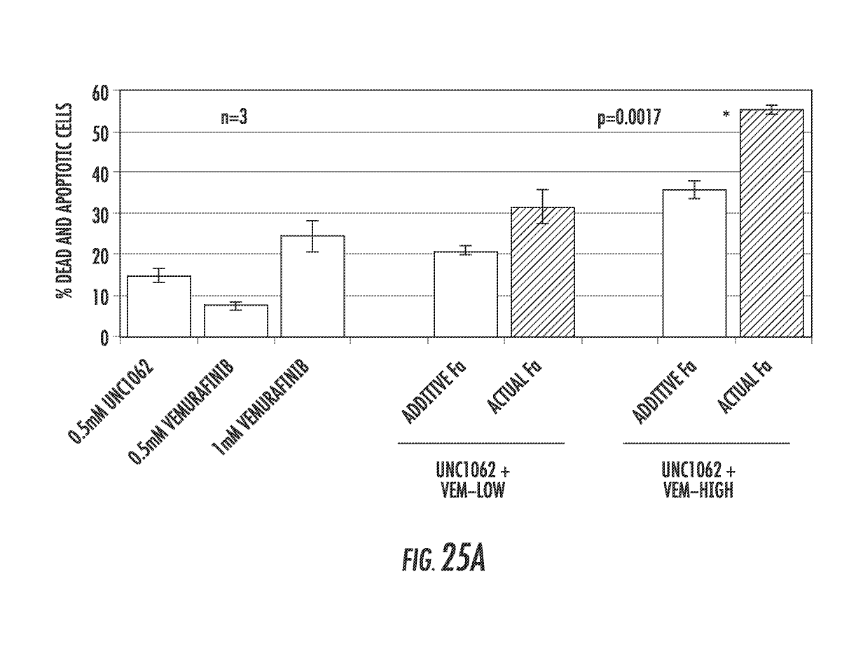

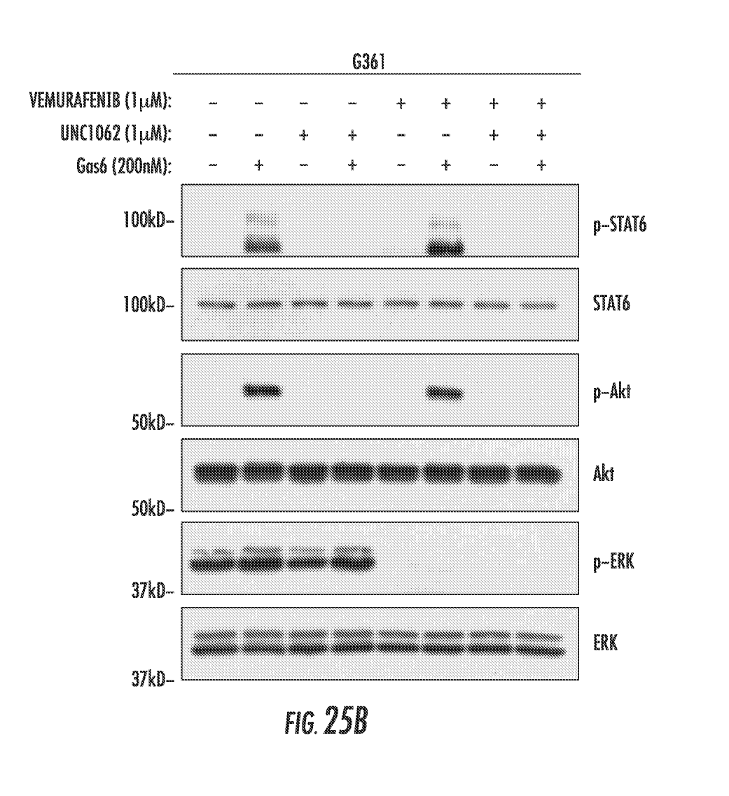

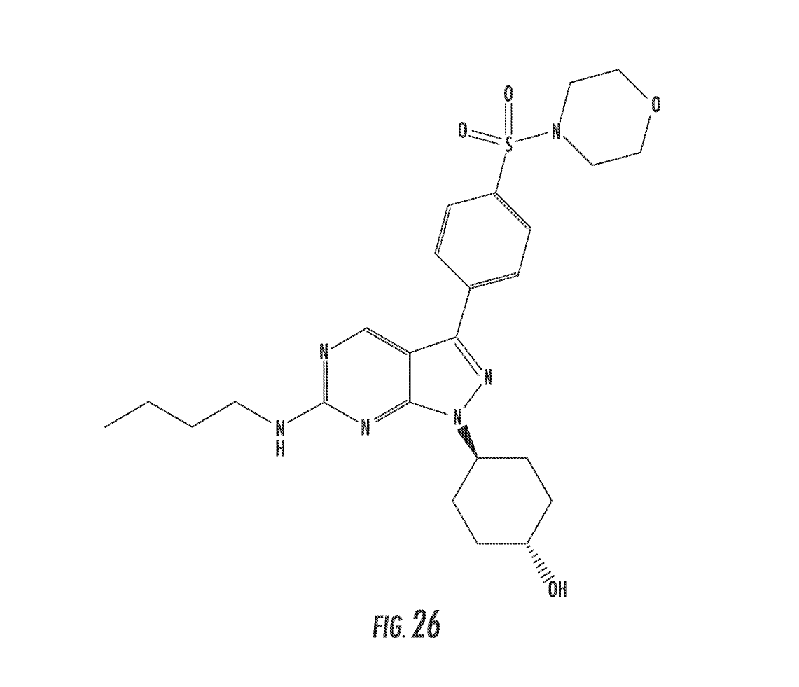

UNC1062, a novel, orally bioavailable and potent MER-selective small-molecule tyrosine kinase inhibitor (TKI) was evaluated in preclinical models of melanoma, both alone and in combination with vemurafenib (a mutant B-RAF TKI). B-RAF wildtype (HMCB) and B-RAF mutant (G361) cell lines were treated with UNC TKI or vehicle. Downstream signaling was evaluated by immunoblotting, and induction of apoptosis was determined by flow cytometry in cells stained with YO-PRO®-1 iodide and propidium iodide. Alternatively, cells were seeded in media containing UNC1062 or vehicle and colony formation was determined. Treatment with MRX6313 induced apoptosis and reduced colony growth in both B-RAF wild-type and B-RAF mutant cell lines, with concentrations as low as 300 nM resulting in an almost complete block in colony formation. In addition, MER inhibition reduced activation of downstream pro-survival signaling pathways known to play roles in melanoma, including ERK, AKT, and STAT6. Importantly, combined treatment with UNC1062 and vemurafenib completely abrogated these signaling pathways in a BRAF mutant cell line and increased apoptosis relative to the single agents, consistent with the idea that MER inhibition may provide additional therapeutic advantage when combined with vemurafenib in patients with B-RAF mutant melanomas. Taken together, these studies validate UNC TKI as a potential treatment for both B-RAF wild-type and B-RAF mutant melanomas and provide data supporting continued development of UNC1062 for treatment of melanoma. See, FIG. 25A and FIG. 25B.

### Example 2

A small molecule Mer tyrosine kinase inhibitor (UNC MerTKi) effectively inhibits growth of murine melanoma.

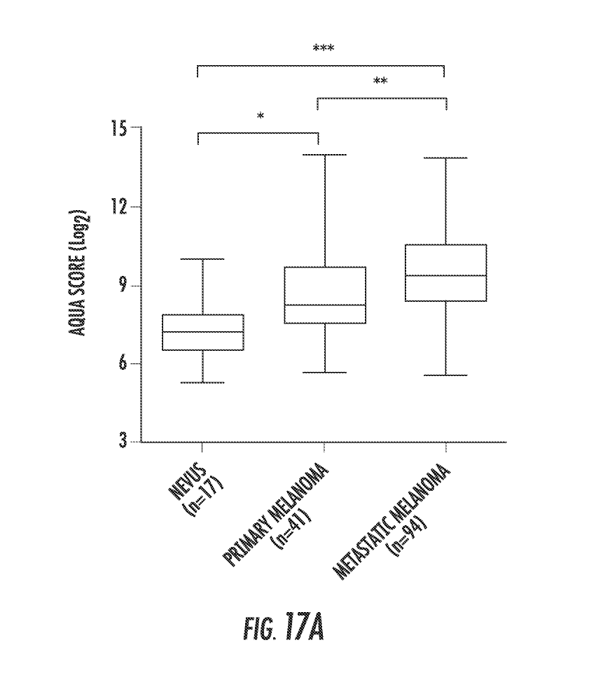

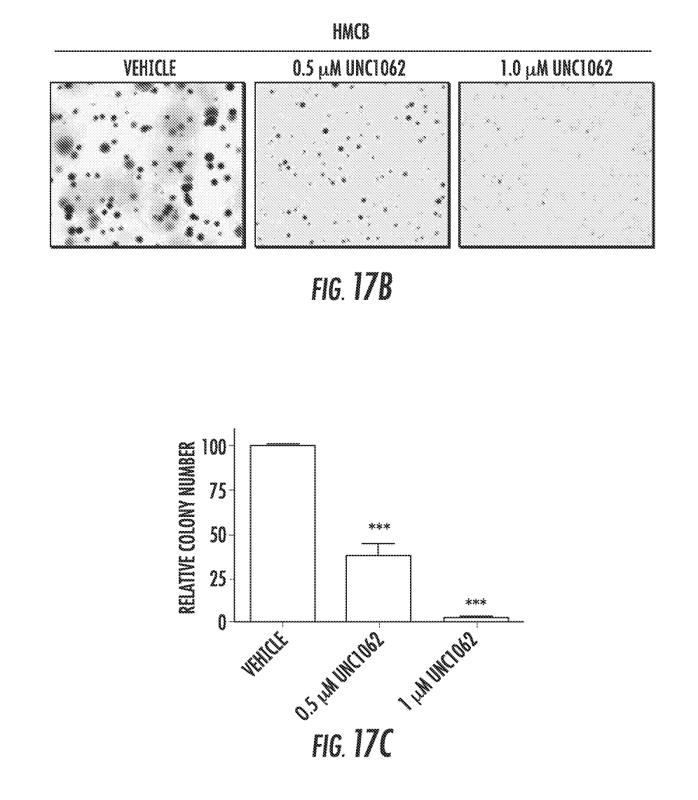

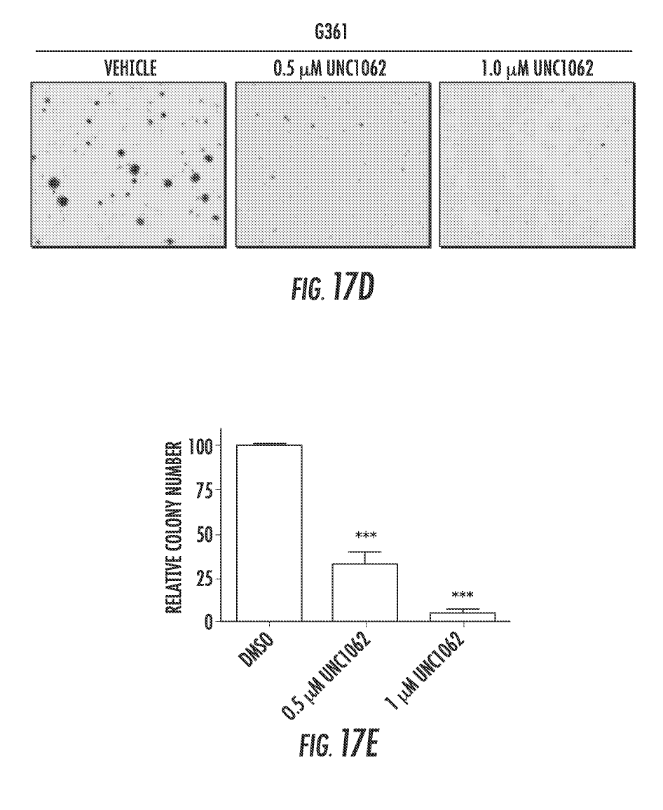

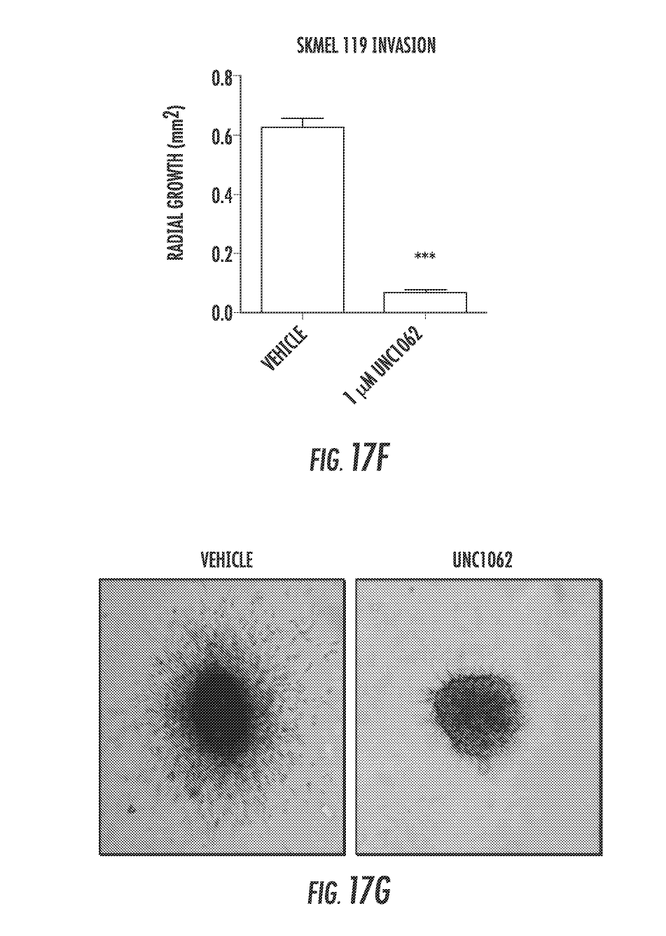

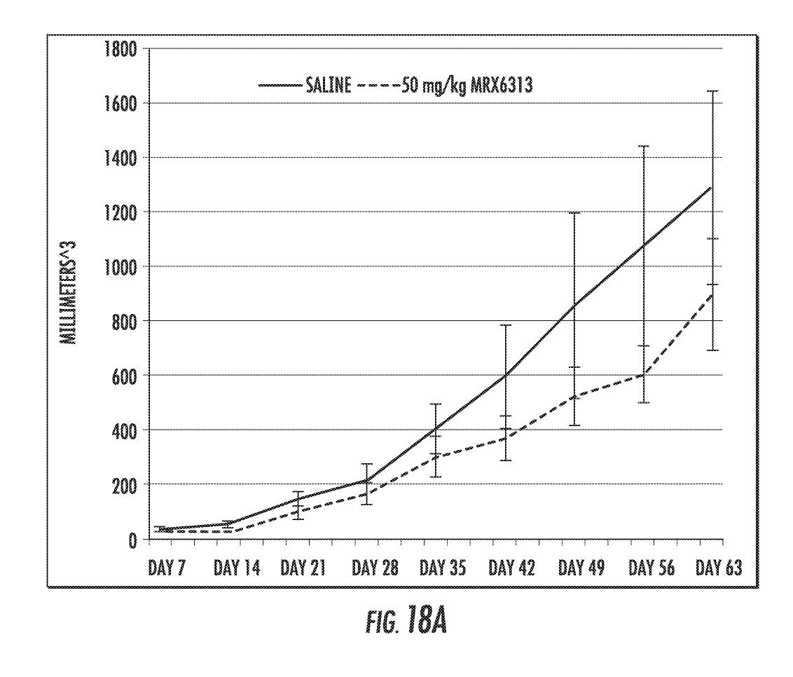

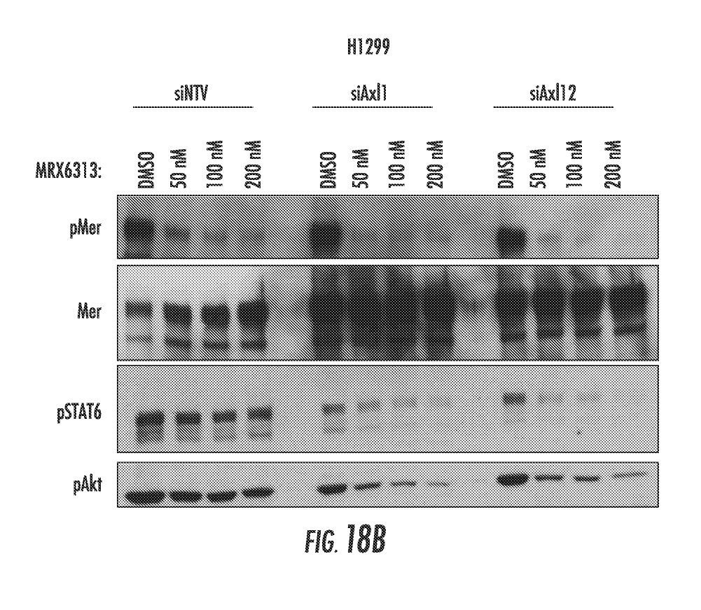

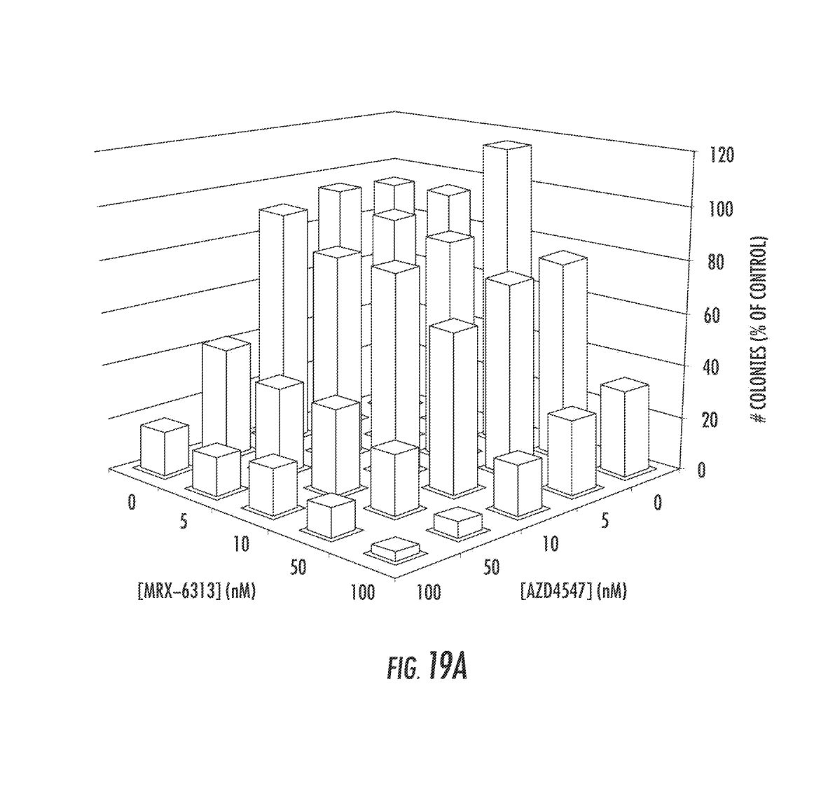

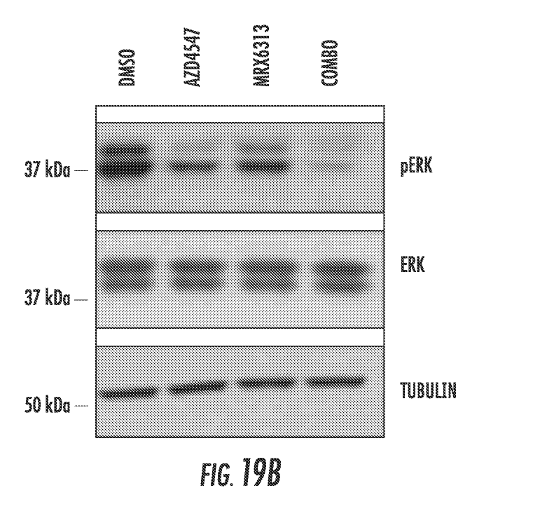

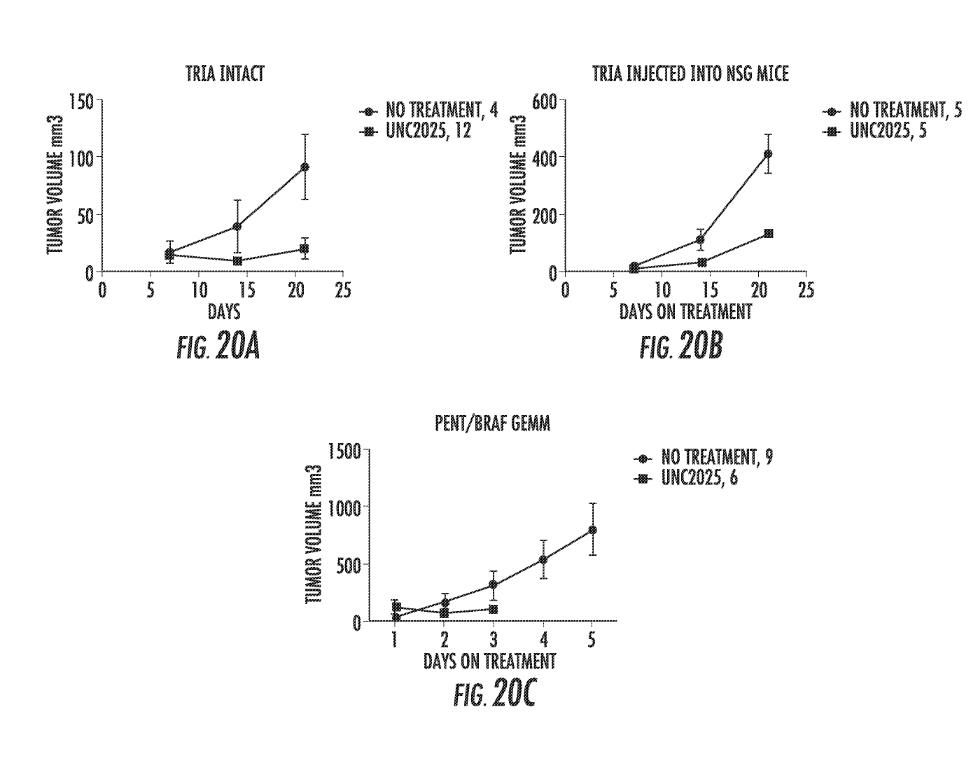

In this example, the activity of first-in-class, orally bioavailable MerTK inhibitor was examined on tumor growth in autochthonous murine tumor models. MRX6313 is 5-fold selective for Mer vs. Axl/Tyro3 and has favorable pharmacokinetics. Once daily, oral dosing inhibits the growth of Mer-expressing leukemia and NSCLC xenografts. MRX6313 was assessed in immune-competent, genetically engineered murine models (GEMMs) in the UNC Lineberger Mouse Phase 1 Unit (MP1U). After dose-finding studies in wild-type mice established an MTD, the inhibitor was given at 120 mpk/d in mouse chow. This dose did not cause weight loss and produced a measurable effect (i.e. inhibition of second phase platelet aggregation, a known Mer pharmacodynamics marker). This dose did not exhibit single agent activity in a murine model of breast cancer (C3TAg), but exhibited pronounced single agent activity in RAS-driven, INK4a/Arf null melanoma GEMM (TRIA). The MP1U has previously reported the efficacy of 15 chemotherapeutic and/or targeted regimens in a large (>220) cohort of TRIA mice (CCR 18:5290, 2012). The overall response was 10% (partial responses and stable disease). There were no complete responses. A combination of MEK (AZD 6244) and PI3K/mTOR (BEZ235) inhibitors were the most active previous regimen (responses seen in 9/18 mice=50%, with 0 CRs) with moderate toxicity. MRX6313 exhibited greater activity (6/8 mice=75%, with 3 CRs) with mild, well tolerated toxicity in the TRIA model. TRIA cell lines do not express Mer, suggesting that MRX6313 as a monotherapy may induce responses via Mer inhibition in TAMs and the tumor microenvironment, or via inhibition of Axl, Tyro or an unknown target. In summary, a potent and selective Mer inhibitor exhibited greater pre-clinical efficacy in a highly faithful model of RAS-mutant melanoma than any other drug tested to date, including several compounds that are FDA approved for use in metastatic melanoma. See, FIG. 16A and FIG. 16B.

### Example 3

A novel Mer tyrosine kinase inhibitor mediates increased cell killing in combination with FGFR inhibition.

In this study the interaction between a novel Mer-selective small molecule tyrosine kinase inhibitor (TKI) (MRX6313) and AZD-4547, an FGFR TKI, in NSCLC cell lines was studied.

### Methods Used:

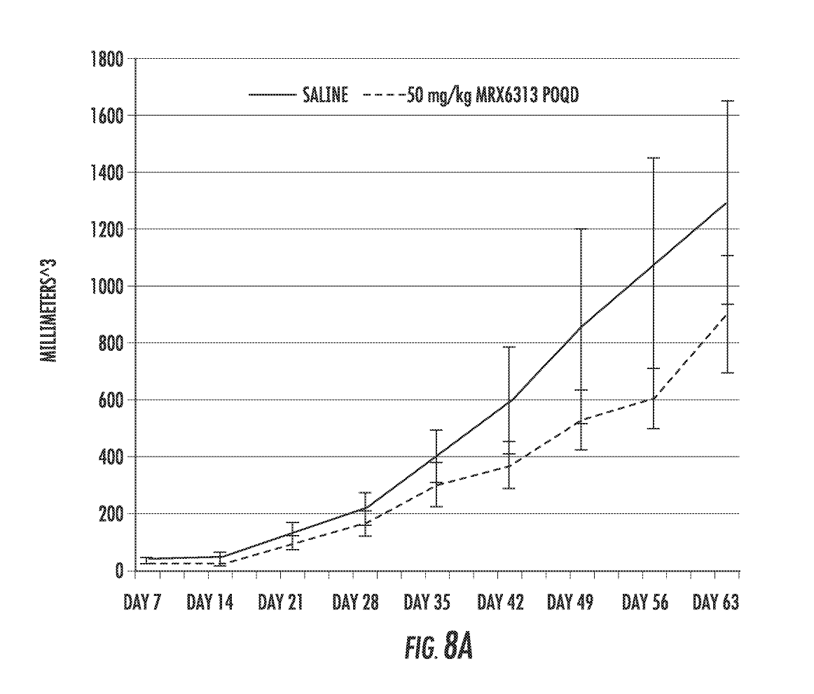

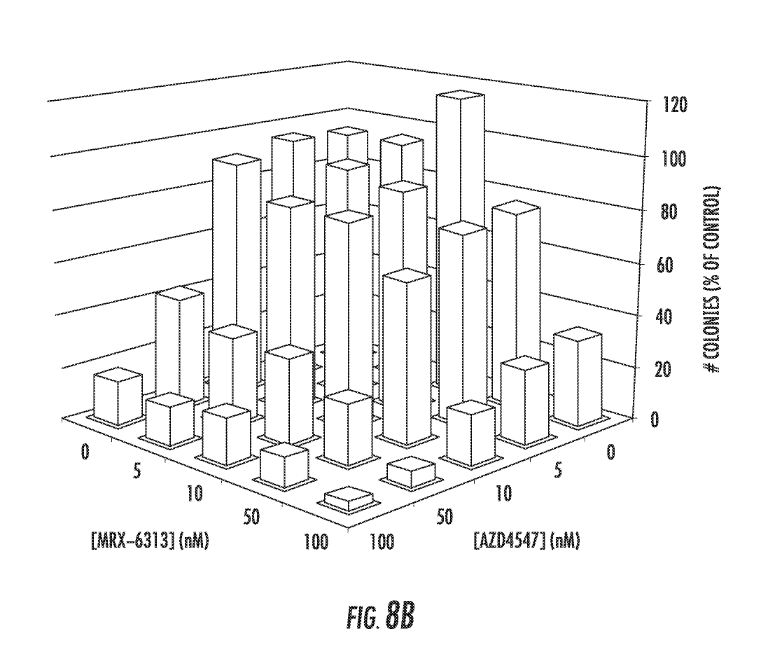

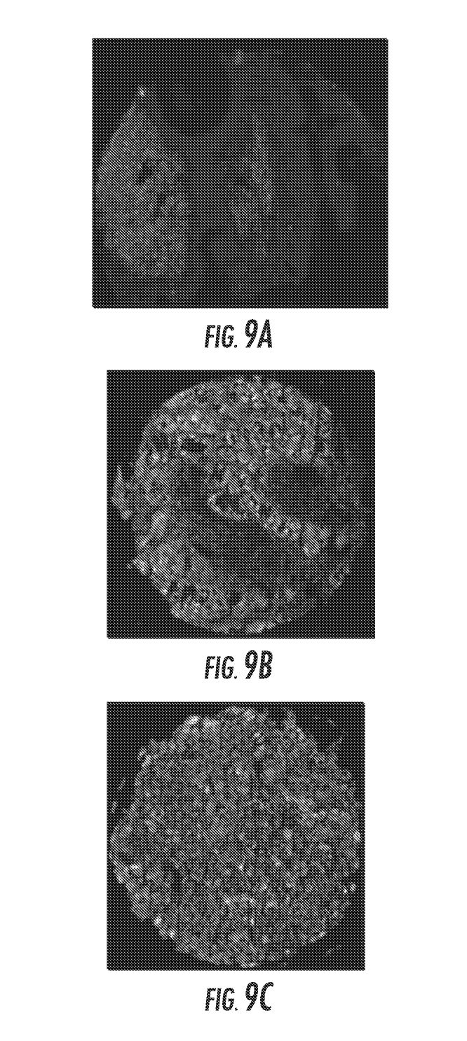

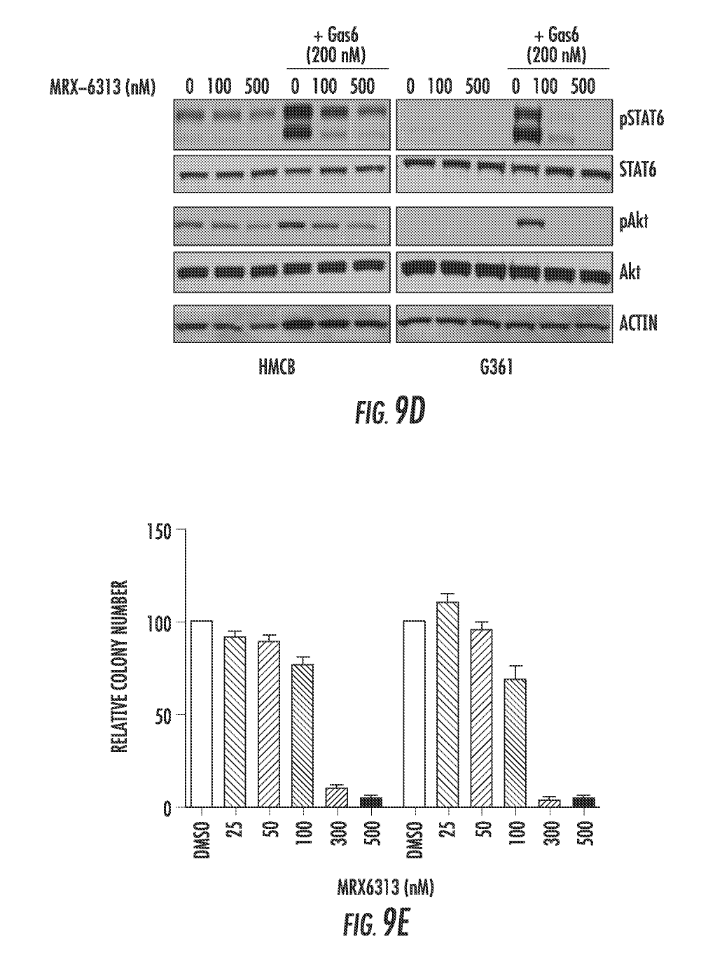

Colo699 (Mer+, FGFR+) and H226 (Mer+, FGFR+) NSCLC cells were cultured for 14 days in soft agar in the presence of MRX6313 and/or AZD-4547, alone or in combination, and colonies were stained and counted. Changes in the activity of downstream signaling pathways, including PI3K/AKT, MEK/ERK, and STAT proteins were evaluated by immunoblotting. In the soft agar assay, Colo699 and H226 colony formation was inhibited in the presence of MRX6313 and AZD-4547, both as single agents and in combination. Importantly, concurrent treatment with Mer TKI and AZD-4547 resulted in a greater decrease in colony-formation relative to either single agent. Immunoblotting revealed increased inhibition of pro-survival signaling in cells treated with both inhibitors relative to the single agents. Taken together, these data suggest that combination therapies targeting Mer kinase and FGFR may be effective for treatment of NSCLC and indicate biochemical mechanisms by which the combination therapy may mediate increased anti-tumor activity. See, FIG. 8A and FIG. 8B.

### Example 4

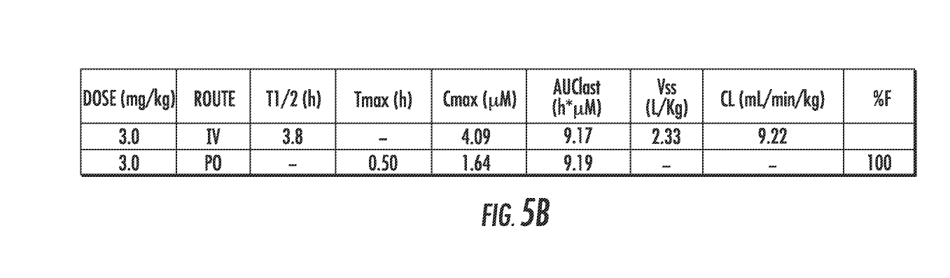

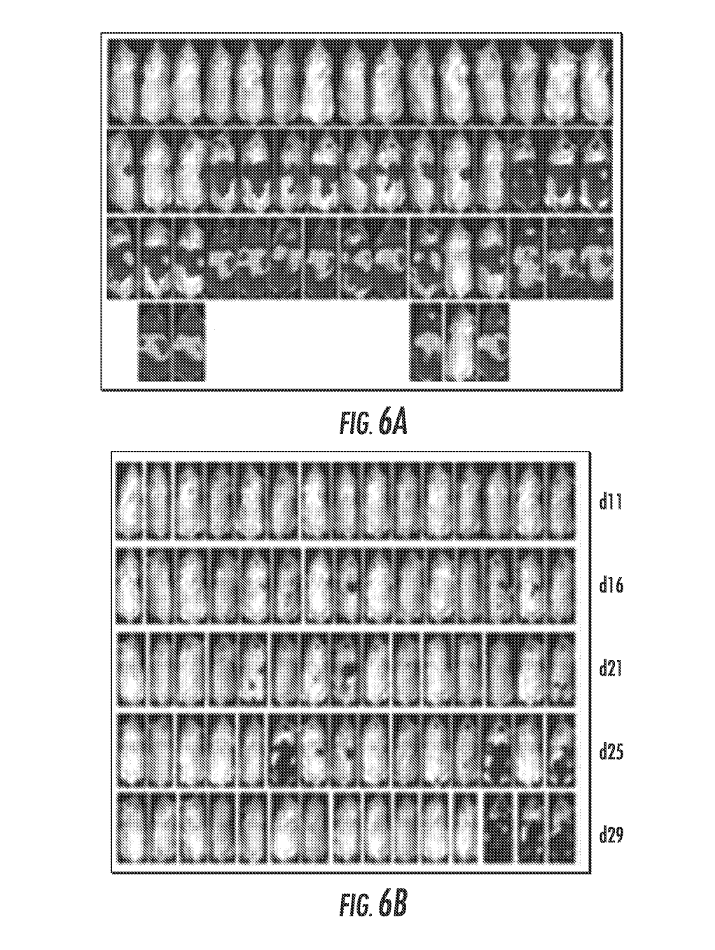

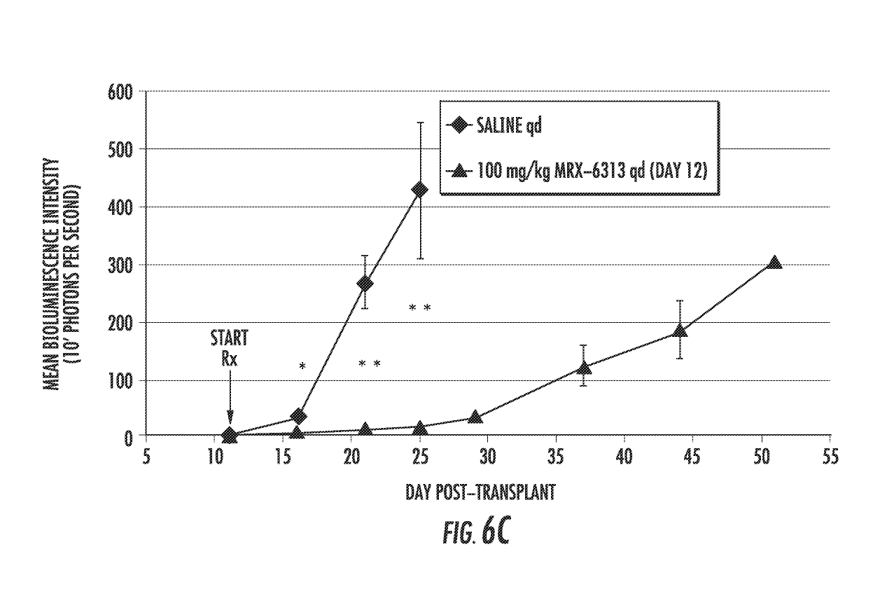

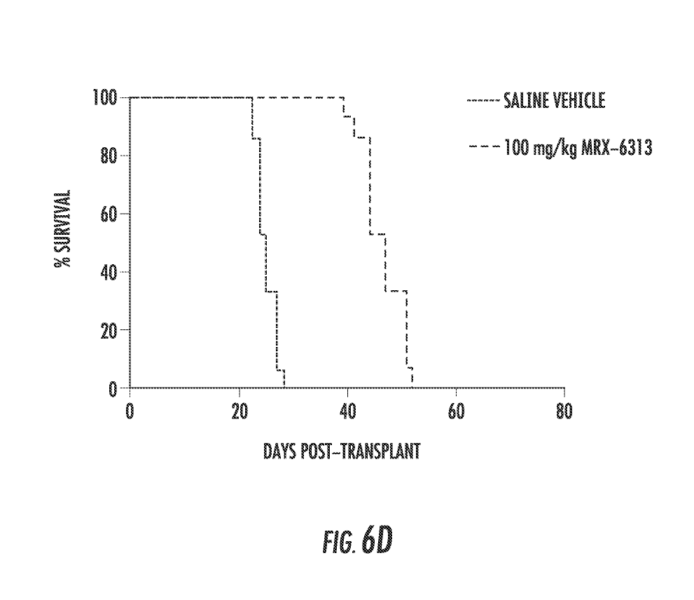

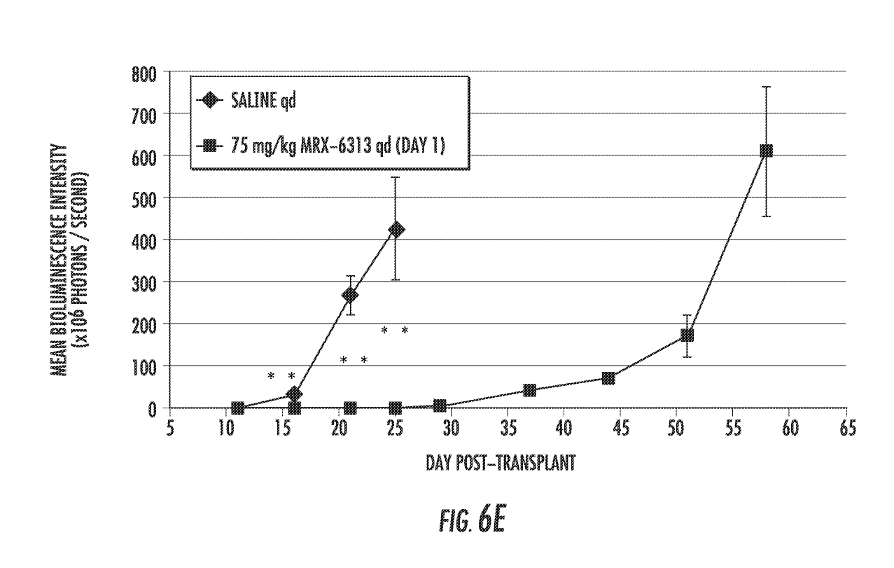

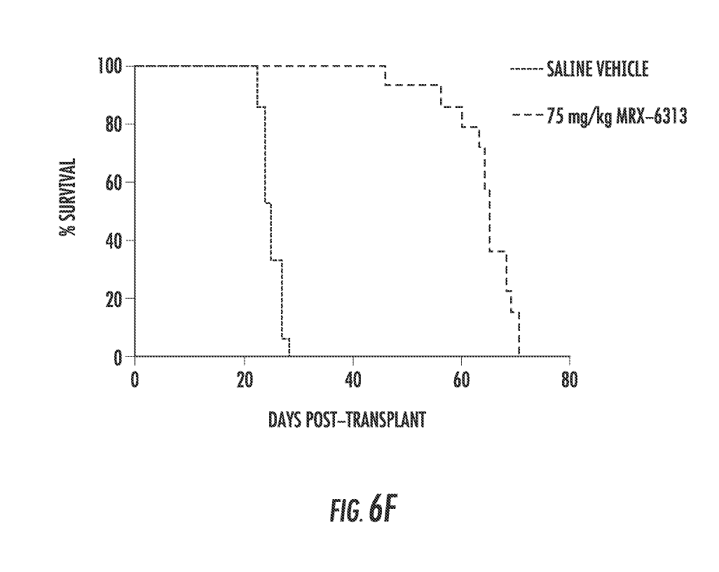

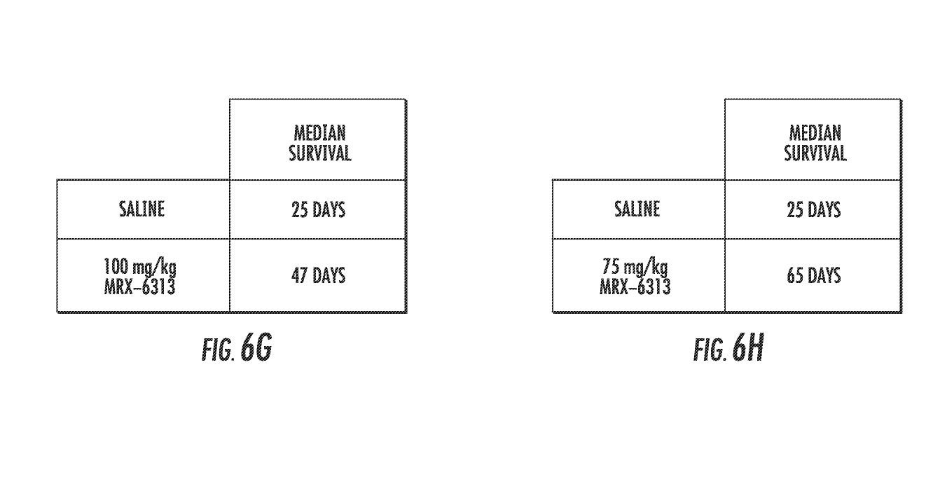

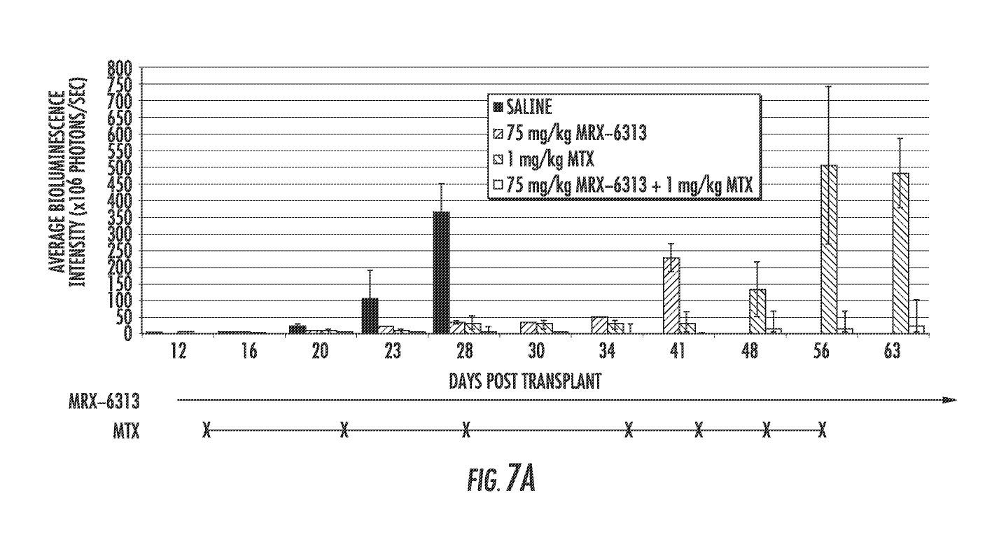

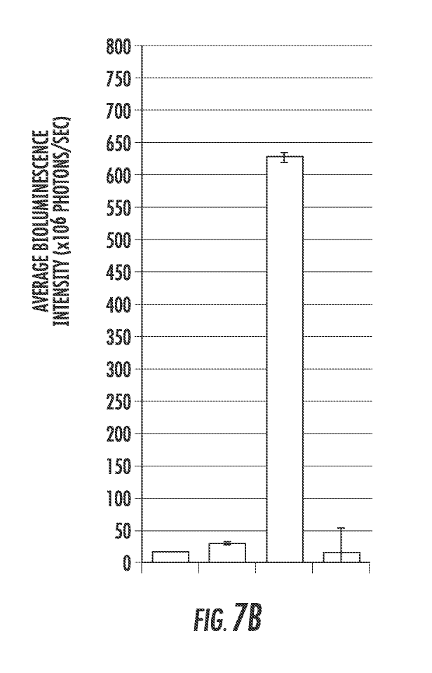

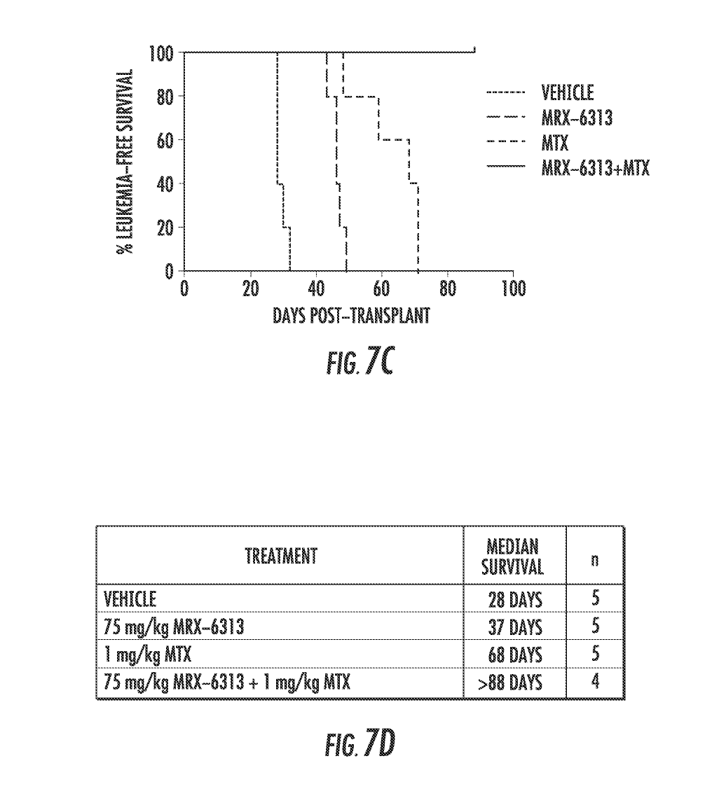

In this example, preclinical testing of a novel, first-in-class MER-selective small molecule tyrosine kinase inhibitor (MRX6313) as a potential therapy for MER-expressing ALL is disclosed. MRX6313 mediates potent inhibition of MER in enzymatic assays (IC₅₀=0.74 nM), has ≧10-fold selectivity for MER over other TAM-family members, and has limited off-target activity against other tyrosine kinases, with the exception of FLT3. In 697 B-ALL cells, MRX6313 inhibited phosphorylation/activation of MER with an IC₅₀ of 2.6 nM and decreased downstream signaling through the ERK and AKT pathways, leading to induction of apoptosis and reduced colony-formation in methylcellulose in MER-expressing ALL cell lines. In mouse models, MRX6313 is orally bioavailable and inhibits MER phosphorylation/activation in leukemic blasts in the bone marrow. In an orthotopic B-ALL xenograft model of minimal residual disease, treatment with MRX6313 resulted in a dose dependent reduction in tumor burden and increased median survival from 27 days after inoculation with tumor cells to 70 days (p<0.0001). In a similar model of existent disease in which leukemia was established for 14 days prior to initiation of treatment, median survival increased from 27.5 to 45 days in response to treatment with MRX6313 (p<0.0001). In both models, tumor burden measured by bioluminescent imaging was significantly decreased in mice treated with MRX6313 relative to mice treated with vehicle, even after the development of advanced disease in the control animals. In addition, treatment with MRX6313 in combination with methotrexate, a chemotherapy that is currently in clinical use for treatment of pediatric ALL, resulted in reduced tumor burden and increased tumor-free survival relative to mice treated with either agent alone. The very high potency, relative selectivity, oral bioavailability, and demonstrated target inhibition and therapeutic efficacy in murine ALL models, both alone and in combination with chemotherapy, identify MRX6313 as an excellent candidate for clinical development in patients with MER-expressing ALL. See, FIGS. 5A-B; 6A-6H; 7A-D.

### Example 5

Inhibition of Mer tyrosine kinase with a novel small molecule inhibitor is efficacious in pre-clinical models of non-small cell lung cancer.

The effects of Mer TKI treatment on activation of Mer and related members of the TAM-family of kinases, Axl and Tyro3, and effects on downstream proliferative and pro-survival signaling pathways were analyzed by immunoblot. In addition, Mer TKI-mediated anti-tumor activity was determined in a panel of NSCLC cell lines using soft-agar and clonogenic assays. Cells were stained with YoPro-1-iodide and propidium iodide dyes and induction of apoptosis was determined using flow cytometry. Finally, a subcutaneous murine xenograft model was employed to determine therapeutic effects in vivo.

Results: The Mer TKI, MRX6313, blocked Mer autophosphorylation in numerous cell lines at sub-micromolar concentrations and was highly selective for Mer over Axl and Tyro3. Treatment also inhibited downstream pro-survival signaling through the ERK1/2 and AKT pathways, which resulted in induction of apoptosis. Additionally, treatment reduced colony-forming potential in soft-agar and clonogenic assays by 85% to 99% in a large panel of cell lines. Sensitivity to the Mer TKI was independent of driver oncogene status, as cell lines positive for EGFR mutations, KRAS mutations, and gene fusions all responded to treatment. Interestingly, RNAi mediated knock-down of Axl enhanced sensitivity to Mer TKI treatment in biochemical and functional assays. Finally, in animals treatment decreased tumor progression resulting in a significant decrease in tumor volume. See FIGS. 8A; 18A-B.

### Example 6

A dual FLT-3 and MER tyrosine kinase small molecule inhibitor in acute myeloid leukemia cell lines and patient samples.

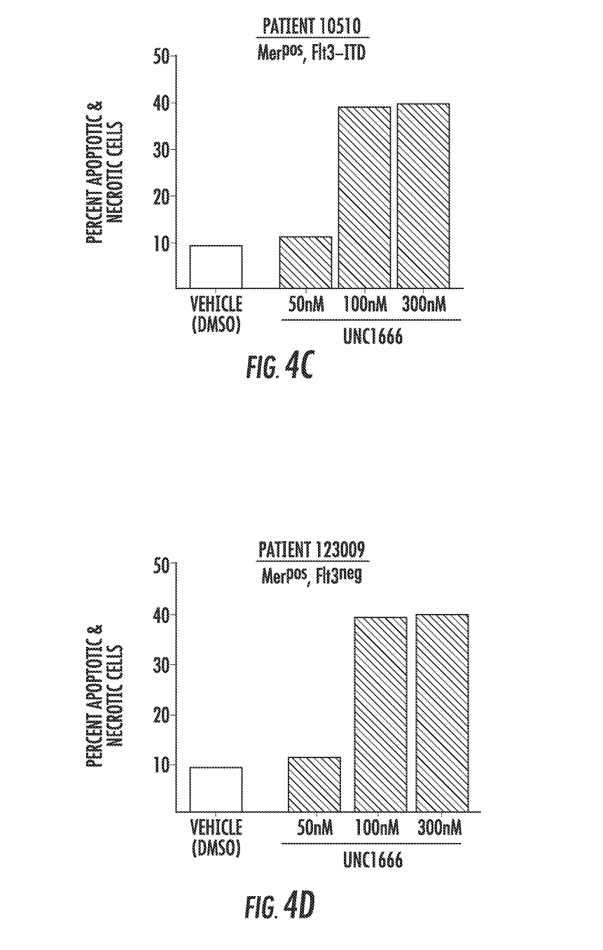

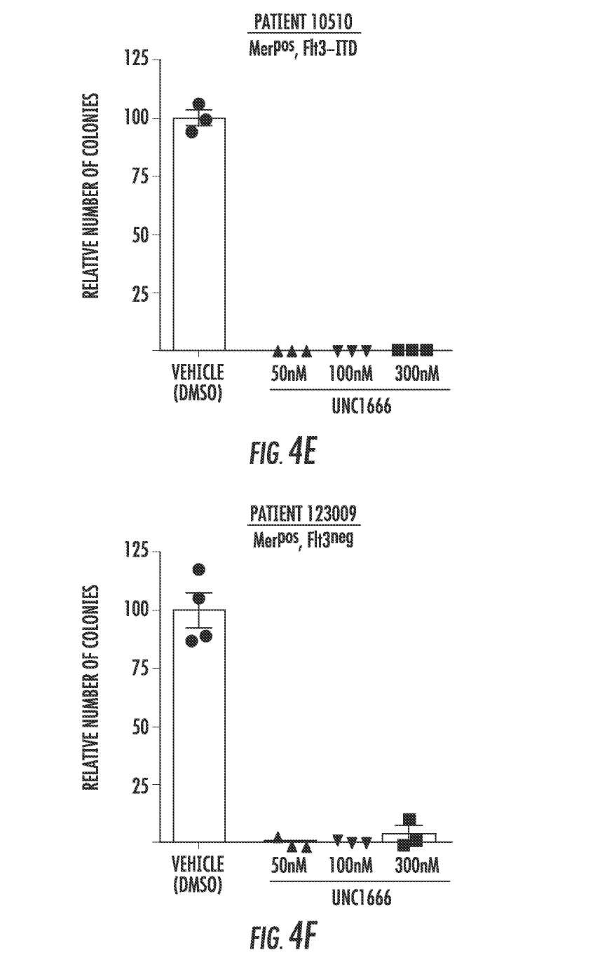

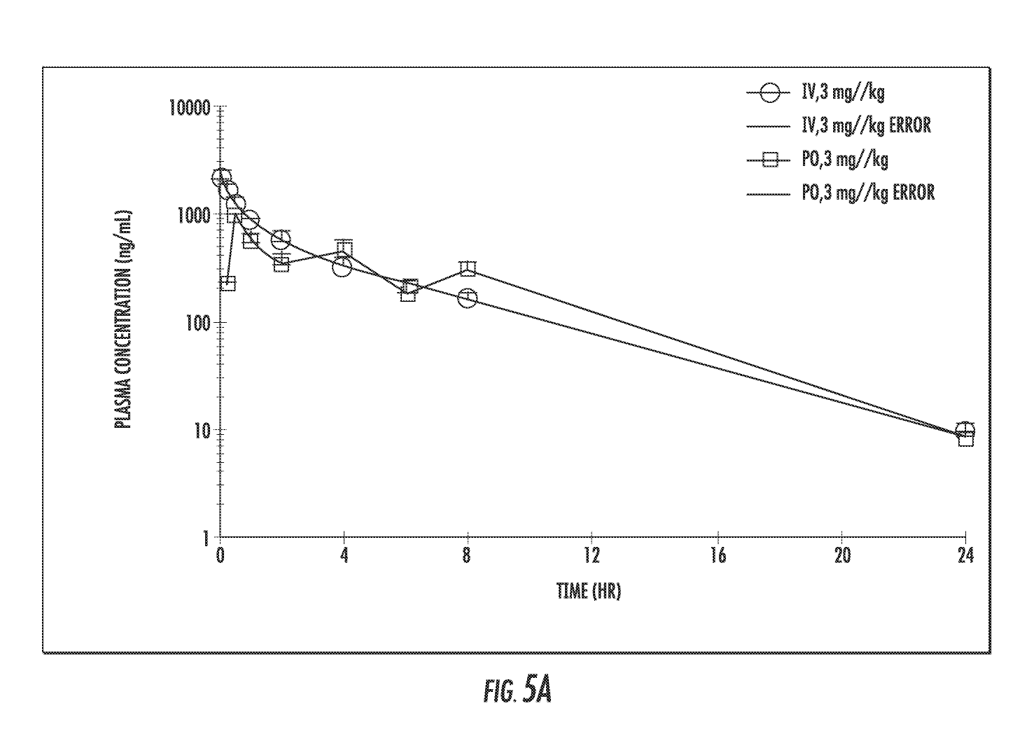

FLT-3 and MER tyrosine kinases have been previously identified as potential targets in the treatment of acute myeloid leukemia (AML). Expression of FLT-3 internal tandem duplication (ITD) occurs in ˜30-40% of AML patient samples and MER overexpression has been detected in ˜80-100%. In this example, a novel first-in-class small molecule inhibitor that has potent activity against both of these kinases and mediates growth inhibition or apoptosis of cell lines and patient myeloblasts is disclosed. UNC1666 is an ATP-competitive reversible small molecule inhibitor that potently inhibits FLT-3 and MER, preventing phosphorylation of these kinases and resultant downstream signaling. In these studies, the effects of treatment with UNC1666 were analyzed in FLT3-ITD-positive (Molm-13 and MV4; 11) and MER-positive (Kasumi-1 and U937) AML cell lines and in primary AML patient samples with variable expression of FLT3-ITD and MER. Short term exposure to UNC1666 in cell lines that express either a FLT3-ITD or MER resulted in a dose-dependent decrease in AKT and STAT6 activation compared to cells treated with vehicle, confirming that UNC1666 inhibits both targets in cell-based assays. AML cell lines were also stained with Yo-Pro-1 iodide and propidium iodide and analyzed by flow cytometry to determine induction of apoptosis in response to treatment with UNC1666. Treatment of MER-positive cell lines with UNC1666 resulted in a two to five-fold induction of apoptosis relative to vehicle-treated cells (66±10% and 20±10% apoptotic cells respectively; p<0.01). Treatment of FLT3-ITD cell lines with UNC1666 resulted in an even more dramatic nine-fold induction of apoptosis (90±6% verses10±2% in vehicle-treated cultures, p<0.001). When AML cell lines were cultured in soft agar, treatment with the dual inhibitor resulted in decreased colony formation compared to cells treated with vehicle (relative colony counts were 100 for vehicle-treated cultures versus, 34±15 for MER-positive cell lines and 15±12 for FLT3-ITD cell lines treated with UNC1666, p<0.01). Six primary patient samples that were MER and/or FLT3-ITD positive were analyzed in similar assays and exhibited dose-dependent induction of apoptosis and near complete inhibition of colony formation in methylcellulose after treatment with UNC1666. See, FIGS. 4A-F.

### Example 7

Targeted inhibition of MER tyrosine kinase in the tumor microenvironment decreases tumor growth in a mouse model of breast cancer.

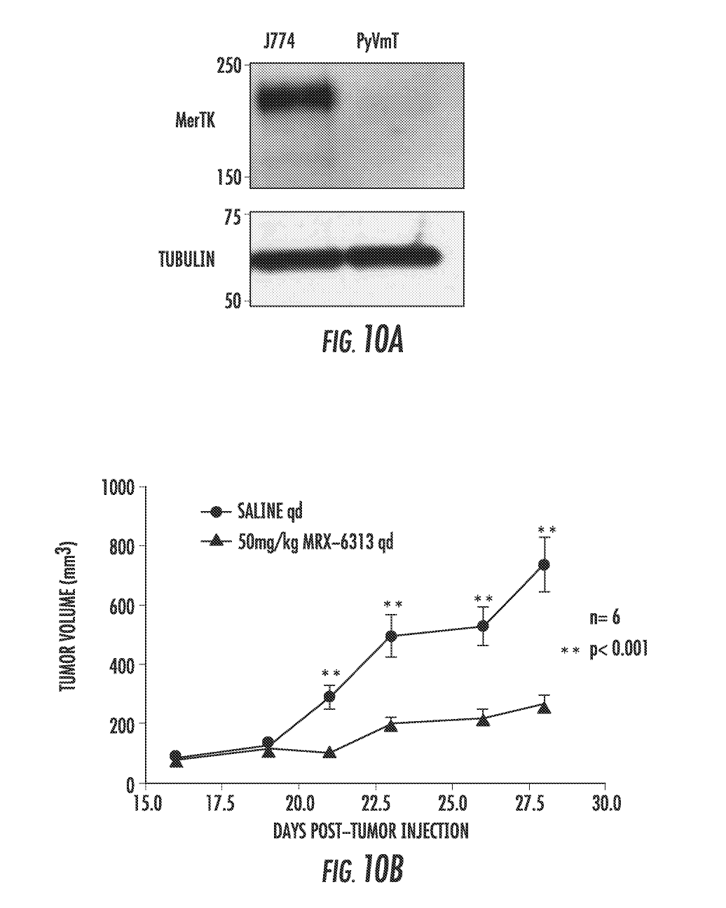

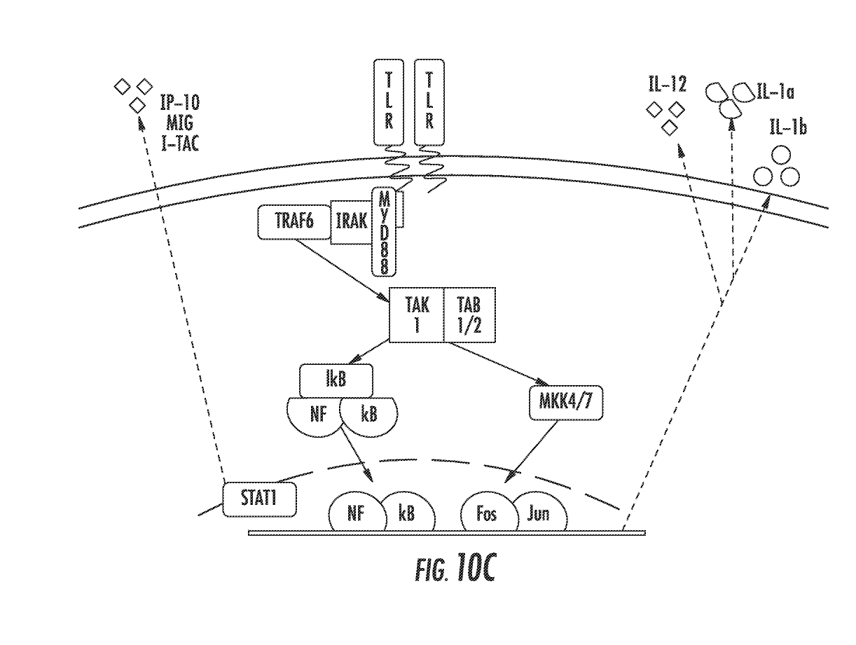

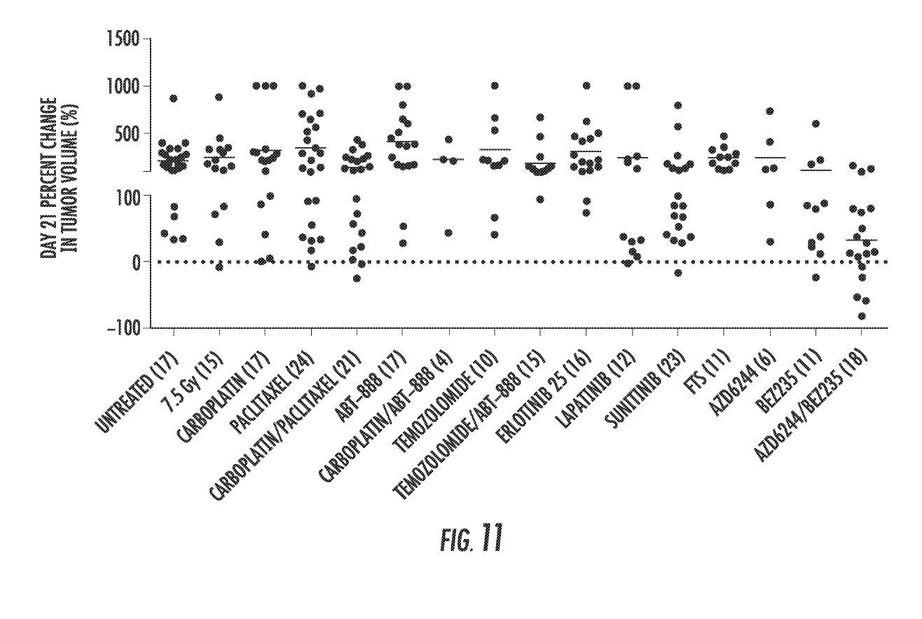

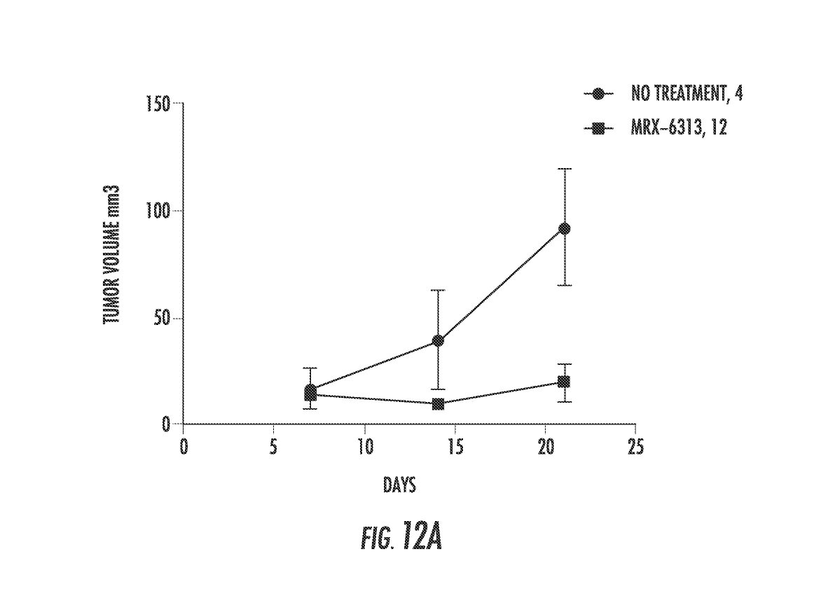

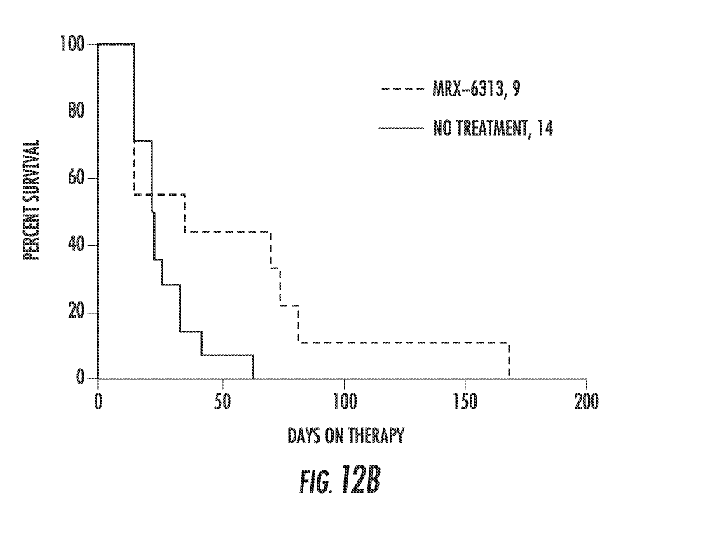

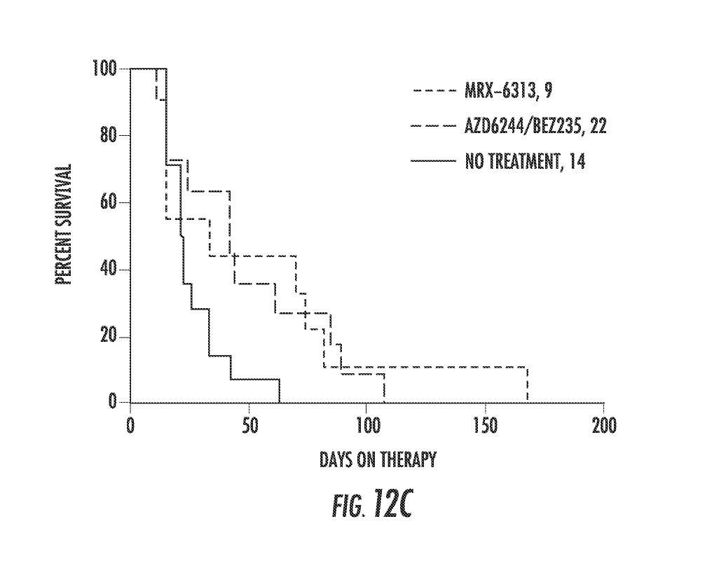

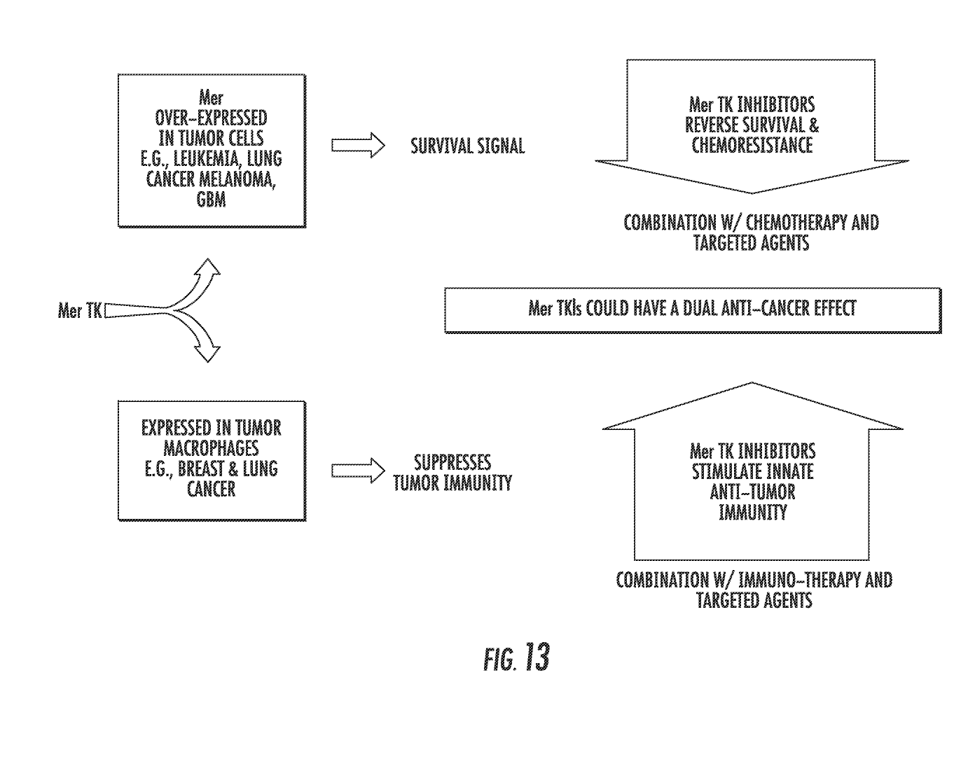

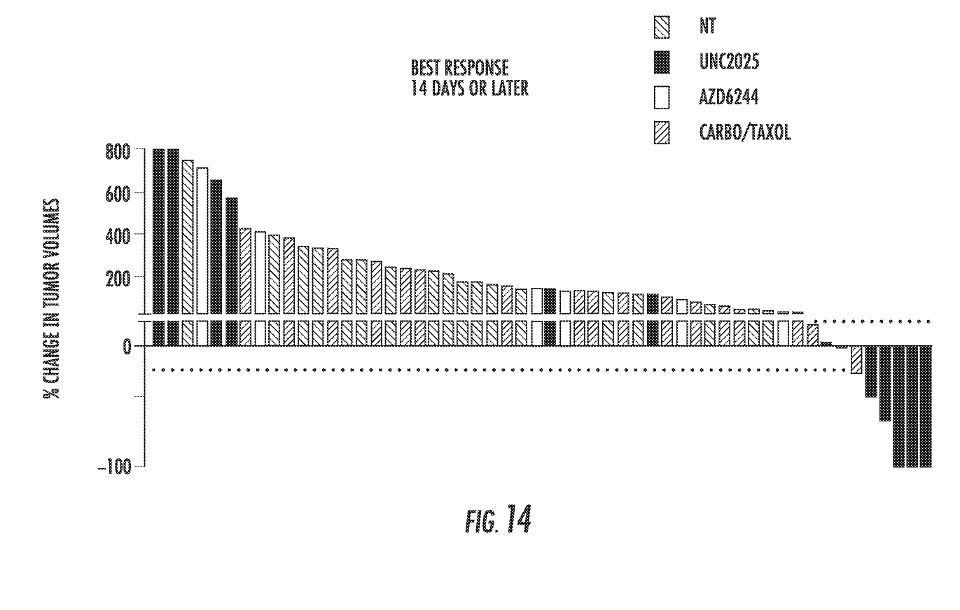

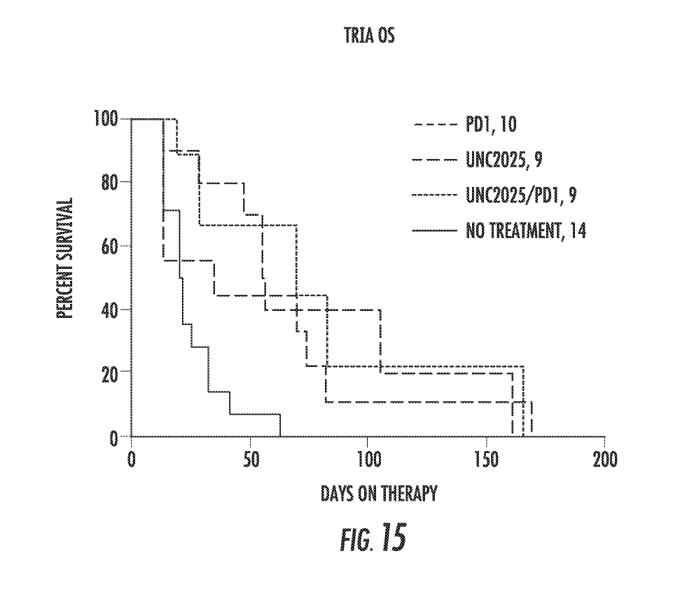

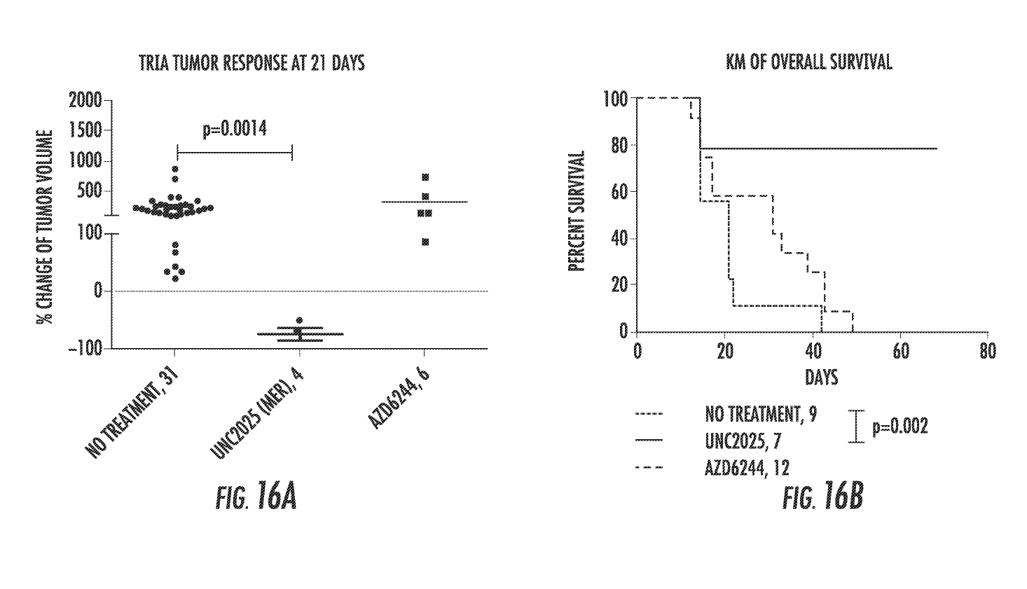

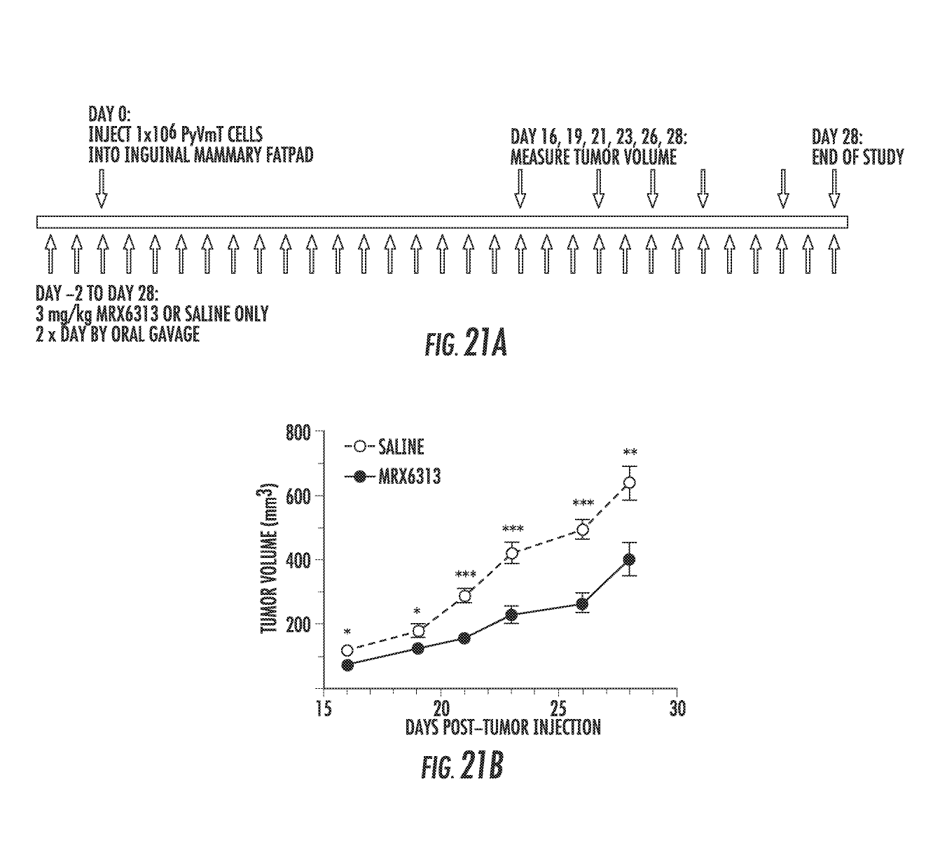

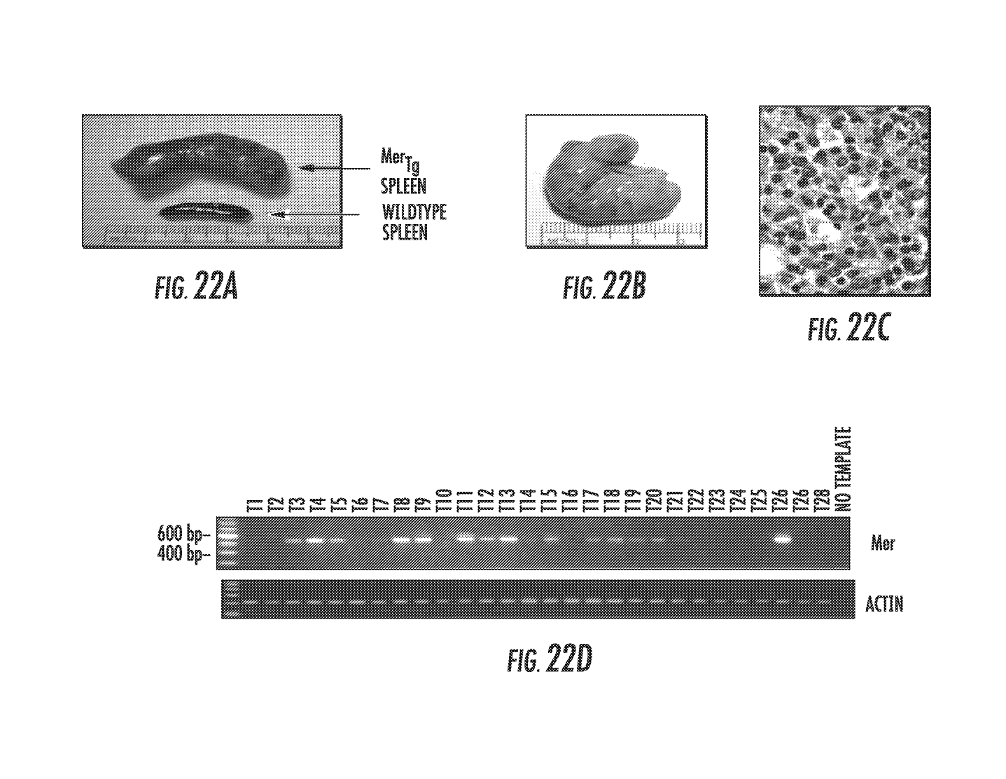

To further investigate the utility of MER inhibition in the tumor microenvironment as a therapeutic strategy, the efficacy of a first in class MER-selective, orally bioavailable, small molecule tyrosine kinase inhibitor (MRX6313) was evaluated in immunocompetent C57Bl/6 mice implanted orthotopically with PyVmT mammary gland tumor cells. These PyVmT tumors cells do not express MER, AXL or TYRO3. Treatment with MRX6313 inhibited phosphorylation of MER in mouse macrophages in vitro, but did not affect survival of macrophages or MER-negative PyVmT tumor cells. However, after four weeks of daily treatment with MRX6313, primary tumor growth was reduced two-fold compared to vehicle-treated tumor bearing mice. Serum IL-10 and IL-4 levels were reduced by 20% and 30%, respectively, in MRX6313 treated tumor-bearing mice compared to vehicle treated tumor-bearing mice. Taken together, these data suggest that MER inhibition in the tumor microenvironment reduces tumor growth by altering the immunosuppressive environment and stimulating anti-tumor immunity. Moreover, these data validate MRX6313 as a promising strategy for immune-mediated treatment of breast cancer. See, FIGS. 10A-10C and FIGS. 21A and 21B.

1. A method of treating a tumor comprising administering an effective amount of a MER tyrosine kinase inhibitor (MER TKI) to inhibit TKI signaling in a tumor.

2. The method of claim 1, wherein inhibition of TKI signaling in the tumor occurs without significantly inhibiting the survival signal of the tumor.

3. The method of claim 1, wherein the method further comprises inhibiting macrophage tumorogenic tolerance during a course of chemotherapy using a conventional tumor chemotherapeutic agent.

4. The method of claim 3, wherein the MER TKI is administered prior to, concurrently with, intermittently, or after chemotherapeutic therapy.

5. The method of claim 3, wherein a low dose of MER TKI is given as an adjunctive therapy with the chemotherapeutic agent.

6. The method of claim 3, wherein administering MER TKI in combination with a chemotherapeutic agent provides for increased anti-tumor effects without an increase in the standard of care dosage of the chemotherapeutic agent.

7. The method of claim 1, wherein the MER TKI is administered as an immunomodulatory agent.

8. The method of claim 1, wherein the tumor is a solid tumor.

9. The method of claim 1, wherein the tumor is a cancer.

10. The method of claim 1, wherein the tumor is selected from the group consisting of a leukemia, a lymphoma, lung cancer, melanoma, breast cancer, pancreatic cancer, glioblastoma and combinations thereof.

11. The method of claim 1, wherein the MER TKI has the following structure:

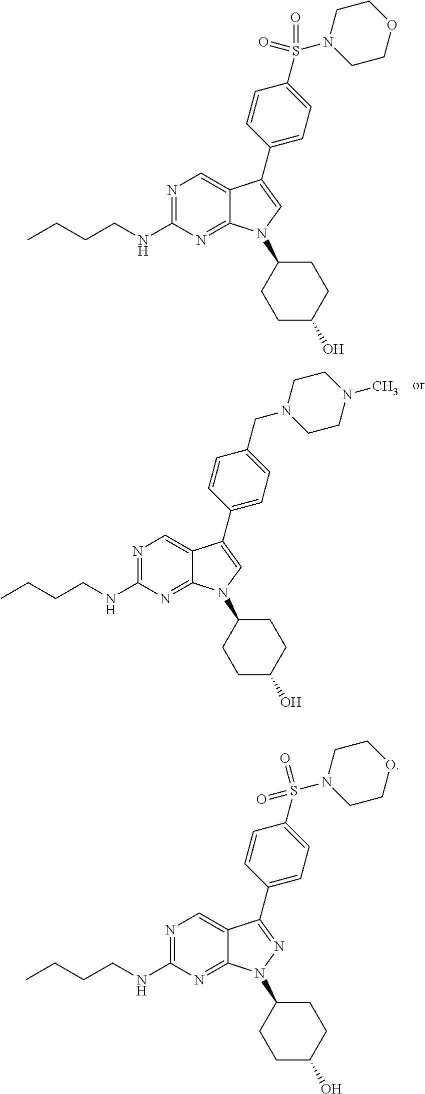

12. The method of claim 3, wherein the chemotherapeutic agent is methotrexate.
